SEQUENCE LISTING (42 sequences)

SEQ ID NO: 1 - protein, 136 aa, synthetic construct
  REGION 1..136: note = T RII-ECD including the structure domain and its natural linkers (also termed T2m)
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 2 - protein, 126 aa, synthetic construct
  REGION 1..126: note = T RII-ECD structured domain with its natural N-terminal linker
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIF

SEQ ID NO: 3 - protein, 111 aa, synthetic construct
  REGION 1..111: note = T RII-ECD structured domain with its with natural C-terminal linker
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 4 - protein, 101 aa, synthetic construct
  REGION 1..101: note = T RII-ECD structured domain
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIF

SEQ ID NO: 5 - protein, 272 aa, synthetic construct
  REGION 1..272: note = T RII-ECD- T RII-ECD fused dimer including structured domains and natural linkers (also termed T22d35)
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 6 - protein, 34 aa, synthetic construct
  REGION 1..34: note = T RII-ECD natural linker
SEEYNTSNPDIPPHVQKSVNNDMIVTDNNGAVKF

SEQ ID NO: 7 - protein, 24 aa, synthetic construct
  REGION 1..24: note = T RII-ECD N-terminal natural linker
IPPHVQKSVNNDMIVTDNNGAVKF

SEQ ID NO: 8 - protein, 10 aa, synthetic construct
  REGION 1..10: note = T RII-ECD C-terminal natural linker
SEEYNTSNPD

SEQ ID NO: 9 - protein, 363 aa, synthetic construct
  REGION 1..363: note = T2m-Fc fusion of T2m with the hIgG2Fc(CCCC) Fc region
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 10 - protein, 499 aa, synthetic construct
  REGION 1..499: note = T22d35-Fc fusion of T22d35 with the hIgG2Fc(CCCC) Fc region
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 11 - protein, 19 aa, synthetic construct
  REGION 1..19: note = signal peptide
MDWTWRILFLVAAATGTHA

SEQ ID NO: 12 - protein, 227 aa, synthetic construct
  REGION 1..227: note = hIgG2Fc(CCCC) Fc region variant
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 13 - protein, 25 aa, synthetic construct
  REGION 1..25: note = T22d35-Fc natural linker with the hIgG2Fc(CCCC) hinge region
SEEYNTSNPDERKCCVECPPCPAPP

SEQ ID NO: 14 - protein, 496 aa, synthetic construct
  REGION 1..496: note = T22d35-Fc-IgG1-v1(CC) fusion of T22d35 with the hIgG1Fc(CC) Fc region
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 224 aa, synthetic construct
  REGION 1..224: note = hIgG1Fc(CC) Fc region variant
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 21 aa, synthetic construct
  REGION 1..21: note = T22d35-Fc-IgG1-v1(CC) natural linker with the hIgG1Fc(CC) hinge region
SEEYNTSNPDTHTCPPCPAPE

SEQ ID NO: 17 - protein, 504 aa, synthetic construct
  REGION 1..504: note = T22d35-Fc-IgG1-v2(SCC) fusion of T22d35 with the hIgG1Fc(SCC) Fc region
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 232 aa, synthetic construct
  REGION 1..232: note = hIgG1Fc(SCC) Fc region variant
VEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 29 aa, synthetic construct
  REGION 1..29: note = T22d35-Fc-IgG1-v2(SCC) natural linker with the hIgG1Fc(SCC) hinge region
SEEYNTSNPDVEPKSSDKTHTCPPCPAPE

SEQ ID NO: 20 - protein, 507 aa, synthetic construct
  REGION 1..507: note = T22d35-Fc-IgG1-v3(GSL-CC) fusion of T22d35 with the hIgG1Fc(CC) Fc region and including an artificial GS linker
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGSGGGSGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - DNA, 11 bp, synthetic construct
  misc_feature 1..11: note = Artificial GS linker of the T22d35-Fc-IgG1-v3(GSL-CC) fusion
  source 1..11: mol_type = other DNA
gggsgggsggg

SEQ ID NO: 22 - protein, 32 aa, synthetic construct
  REGION 1..32: note = T22d35-Fc-IgG1-v3(GSL-CC) linker including natural and artificial sequences and hIgG1Fc(CC) hinge region
SEEYNTSNPDGGGSGGGSGGGTHTCPPCPAPE

SEQ ID NO: 23 - protein, 494 aa, synthetic construct
  REGION 1..494: note = T22d35-Fc-IgG2-v2(CC) fusion of T22d35 with the hIgG2Fc(CC) Fc region
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 24 - protein, 222 aa, synthetic construct
  REGION 1..222: note = hIgG2Fc(CC) Fc region variant
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 25 - protein, 20 aa, synthetic construct
  REGION 1..20: note = T22d35-Fc-IgG2-v2(CC) natural linker with the hIgG2Fc(CC) hinge region
SEEYNTSNPDVECPPCPAPP

SEQ ID NO: 26 - protein, 492 aa, synthetic construct
  REGION 1..492: note = hIgG1Fc?K(C)-T22d35
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 27 - protein, 498 aa, synthetic construct
  REGION 1..498: note = hIgG1Fc?K(CC)-T22d35
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 28 - protein, 494 aa, synthetic construct
  REGION 1..494: note = hIgG2Fc?K(CC)-T22d35
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 29 - DNA, 1560 bp, synthetic construct
  misc_feature 1..1560: note = Nucleic acid sequence encoding T22d35-Fc in secretable form
  source 1..1560: mol_type = other DNA
atggattggacctggagaatcctcttccttgtagcagcagcaacaggtacacatgctatccctcctcatgttcaaaagtccgttaacaacgacatgatcgtcaccgataacaacggtgctgtcaagttcccacaactctgtaagttctgcgatgtgcgtttctccacatgtgataaccagaagtcctgtatgagcaactgctcaatcacctccatctgcgaaaagccacaagaggtatgcgtagctgtatggcgaaagaacgatgaaaacatcaccctggaaaccgtctgtcacgatccaaagctcccataccatgatttcatcctggaagacgcagcttctccaaagtgtatcatgaaggagaagaagaagcccggtgaaaccttcttcatgtgctcctgttcctcagatgaatgcaacgataacatcatcttctccgaggagtacaacacctccaacccagatatccctccacacgttcagaagtccgtaaacaatgacatgattgtgaccgacaacaacggggctgttaagttcccacagctctgtaagttttgcgacgttaggttcagcacctgtgataatcagaagagctgcatgtccaactgcagcatcaccagtatttgcgagaagcctcaagaagtgtgtgtcgctgtttggagaaagaacgacgaaaacataaccctggagaccgtttgccacgatccaaaactcccatatcacgatttcattctggaggacgccgccagtcctaaatgtataatgaaagagaagaagaaaccaggggagaccttctttatgtgcagctgcagcagcgacgagtgtaacgataatataatttttagcgaggagtataatacaagcaatcccgacgagcgcaagtgctgcgtcgagtgccctccatgccctgcccctcctgttgccggacctagtgtgtttttgtttcctcctaaacctaaagatacactcatgattagcaggacacctgaggtgacatgtgtcgtcgtggacgtgagtcatgaagaccccgaagtgcagtttaattggtatgtcgacggagtcgaagtccataatgccaaaactaaaccaagggaagaacagtttaattcaacttttcgcgtggtctctgtgctgactgtggtgcaccaggactggcttaatggaaaggaatacaagtgtaaggtgagtaataagggcctgcccgcccccattgaaaaaactattagtaagactaaagggcagccccgagagccccaggtgtatactttgcccccctctcgggaggagatgactaaaaatcaggtgagtcttacatgtcttgtgaaaggattttacccctctgacatttcagtggagtgggagtctaatggccagcccgagaataattacaaaactactccccccatgttggactctgacggctcatttttcttgtactctaaactgacagtggacaaaagtcggtggcagcagggcaatgtgttttcttgttcagtgatgcacgaggccctgcataatcactatacacagaaatctctgtctctgtcacccggctgatga

SEQ ID NO: 30 - DNA, 1545 bp, synthetic construct
  misc_feature 1..1545: note = Nucleic acid sequence encoding T22d35-Fc-IgG1-v1(CC) in secretable form
  source 1..1545: mol_type = other DNA
atggactggacctggagaatcctgttcctggtggctgctgctaccggaacacacgctatcccccctcatgtgcagaagtccgtgaacaatgacatgatcgtgacagataacaatggcgccgtgaagtttcctcagctgtgcaagttctgtgacgtgaggtttagcacctgcgataaccagaagtcctgcatgagcaattgttctatcacatccatctgcgagaagccacaggaggtgtgcgtggccgtgtggcggaagaacgacgagaatatcaccctggagacagtgtgccacgatcctaagctgccataccatgacttcatcctggaggatgctgcctctcccaagtgtatcatgaaggagaagaagaagcctggcgagacattcttcatgtgctcctgttccagcgacgagtgcaacgataatatcatcttcagcgaggagtataacacctctaatccagatatcccaccccacgtgcagaagtctgtcaataacgatatgattgtcacagataacaatggcgctgtgaagtttccccagctgtgcaaattttgtgacgtgagattttccacctgtgataaccagaagagctgcatgtctaattgttccatcacatctatttgtgaaaaacctcaggaagtgtgcgtggccgtgtggagaaaaaatgatgaaaacatcaccctggagacagtgtgccatgatcccaagctgccttatcacgacttcatcctggaagacgctgccagcccaaaatgcattatgaaagagaagaagaagcccggtgagacattcttcatgtgcagctgttcttctgatgaatgtaacgataatatcatcttttccgaggagtataacacaagcaatcccgacacccacacatgccctccatgtccagctcctgagctgctgggaggacctagcgtgttcctgtttccccctaagccaaaggataccctgatgatcagcaggacccccgaggtgacatgcgtggtggtggacgtgtctcacgaggaccccgaggtgaagtttaactggtacgtggacggcgtggaggtgcataatgccaagaccaagcctagggaggagcagtacaactctacctatcgggtggtgtccgtgctgacagtgctgcatcaggattggctgaacggcaaggagtataagtgcaaggtgtccaataaggctctgccagcccccattgagaagaccatcagcaaggctaagggccagccaagagagccccaggtgtacacactgccaccctctcgcgacgagctgaccaagaaccaggtgtccctgacatgtctggtgaagggcttctatccttccgatatcgctgtggagtgggagagcaacggacagccagagaacaattacaagaccacacctccagtgctggactctgatggctccttctttctgtatagcaagctgaccgtggacaagtctaggtggcagcagggcaacgtgtttagctgttctgtgatgcatgaggccctgcacaatcattacacacagaagtccctgagcctgtctcctggc

SEQ ID NO: 31 - DNA, 1569 bp, synthetic construct
  misc_feature 1..1569: note = Nucleic acid sequence encoding T22d35-Fc-IgG1-v2(SCC) in secretable form
  source 1..1569: mol_type = other DNA
atggactggacctggagaatcctgttcctggtggctgctgctaccggaacacacgctatcccccctcatgtgcagaagtctgtgaacaatgacatgatcgtgacagataacaatggcgccgtgaagtttccccagctgtgcaagttctgtgacgtgaggttttccacctgcgataaccagaagtcttgcatgtccaattgtagcatcacatctatctgcgagaagcctcaggaggtgtgcgtggccgtgtggcggaagaacgacgagaatatcaccctggagacagtgtgccacgatcctaagctgccataccatgacttcatcctggaggatgctgccagcccaaagtgtatcatgaaggagaagaagaagcccggcgagacattcttcatgtgctcttgttccagcgacgagtgcaacgataatatcatcttctccgaggagtataacaccagcaatcctgacatcccaccccacgtgcagaagagcgtcaataacgatatgattgtcacagataacaatggcgctgtgaagtttccacagctgtgcaaattttgtgacgtgagattttctacctgtgataaccagaagtcctgcatgagcaattgttctatcacatccatctgcgagaagccacaggaagtgtgcgtggccgtgtggagaaaaaatgatgaaaacatcaccctggagacagtgtgccatgatcccaagctgccttatcacgacttcatcctggaagacgctgcctcccctaaatgcattatgaaagagaagaagaagccaggtgagacattcttcatgtgcagctgttcttctgatgagtgcaacgataacatcatcttttctgaggagtacaacacatccaatcctgacgtggagccaaagagctctgataagacccacacatgccctccatgtccagctcctgagctgctgggaggaccatccgtgttcctgtttccacctaagcctaaggacaccctgatgatctccaggaccccagaggtgacatgcgtggtggtggacgtgagccacgaggaccccgaggtgaagtttaactggtacgtggatggcgtggaggtgcataatgccaagaccaagccaagggaggagcagtacaacagcacctatcgggtggtgtctgtgctgacagtgctgcatcaggactggctgaacggcaaggagtataagtgcaaggtgtctaataaggctctgccagcccccatcgagaagaccatctccaaggctaagggccagccaagagagccccaggtgtacacactgccacccagccgcgacgagctgaccaagaaccaggtgtctctgacatgtctggtgaagggcttctatccctctgatatcgctgtggagtgggagtccaacggacagcctgagaacaattacaagaccacacctccagtgctggacagcgatggctctttctttctgtattccaagctgaccgtggataagagcaggtggcagcagggcaacgtgttttcctgtagcgtgatgcatgaggccctgcacaatcattacacacagaagtctctgtccctgagccctggc

SEQ ID NO: 32 - DNA, 1578 bp, synthetic construct
  misc_feature 1..1578: note = Nucleic acid sequence encoding T22d35-Fc-IgG1-v3(GSL-CC) in secretable form
  source 1..1578: mol_type = other DNA
atggattggacctggagaatcctgttcctggtggctgctgctaccggaacacacgctatcccccctcatgtgcagaagtctgtgaacaatgacatgatcgtgacagataacaatggcgccgtgaagtttcctcagctgtgcaagttctgtgacgtgaggttttccacctgcgataaccagaagtcctgcatgagcaattgttctatcacatccatctgcgagaagccacaggaggtgtgcgtggccgtgtggcggaagaacgacgagaatatcaccctggagacagtgtgccacgatcctaagctgccataccatgacttcatcctggaggatgctgccagccccaagtgtatcatgaaggagaagaagaagcctggcgagacattcttcatgtgctcttgttccagcgacgagtgcaacgataatatcatcttctccgaggagtataacaccagcaatccagacatcccaccccacgtgcagaagagcgtcaataacgatatgattgtcacagataacaatggcgctgtgaagtttccccagctgtgcaaattttgtgacgtgagattttctacctgtgataaccagaagagctgcatgtctaattgttccatcacatctatttgtgaaaaacctcaggaagtgtgcgtggccgtgtggagaaaaaatgatgaaaacatcaccctggagacagtgtgccatgatcccaagctgccttatcacgacttcatcctggaagacgctgcctccccaaaatgcattatgaaagagaagaagaagcccggtgagacattcttcatgtgcagctgttcttctgatgagtgcaacgataacatcatcttttctgaggagtacaacacatccaatcctgacggaggaggcagcggaggaggctctggaggcggcacccacacatgccctccatgtccagctcctgagctgctgggaggaccttccgtgttcctgtttccccctaagccaaaggacaccctgatgatctccaggacccccgaggtgacatgcgtggtggtggacgtgagccacgaggaccccgaggtgaagtttaactggtacgtggatggcgtggaggtgcataatgccaagaccaagccaagggaggagcagtacaacagcacctatcgggtggtgtctgtgctgacagtgctgcatcaggattggctgaacggcaaggagtataagtgcaaggtgtctaataaggctctgccagcccccattgagaagaccatctccaaggctaagggccagccaagagagccccaggtgtacacactgccacccagccgcgacgagctgaccaagaaccaggtgtctctgacatgtctggtgaagggcttctatccttctgatatcgctgtggagtgggagtccaacggacagccagagaacaattacaagaccacacctccagtgctggactctgatggctccttctttctgtattccaagctgaccgtggacaagagcaggtggcagcagggcaacgtgtttagctgttctgtgatgcatgaggccctgcacaatcattacacacagaagtccctgagcctgtctcctggc

SEQ ID NO: 33 - DNA, 1545 bp, synthetic construct
  misc_feature 1..1545: note = Nucleic acid sequence encoding T22d35-Fc-IgG2-v2(CC) in secretable form
  source 1..1545: mol_type = other DNA
atggattggacctggagaatcctcttccttgtagcagcagcaacaggtacacatgctatccctcctcatgttcaaaagtccgttaacaacgacatgatcgtcaccgataacaacggtgctgtcaagttcccacaactctgtaagttctgcgatgtgcgtttctccacatgtgataaccagaagtcctgtatgagcaactgctcaatcacctccatctgcgaaaagccacaagaggtatgcgtagctgtatggcgaaagaacgatgaaaacatcaccctggaaaccgtctgtcacgatccaaagctcccataccatgatttcatcctggaagacgcagcttctccaaagtgtatcatgaaggagaagaagaagcccggtgaaaccttcttcatgtgctcctgttcctcagatgaatgcaacgataacatcatcttctccgaggagtacaacacctccaacccagatatccctccacacgttcagaagtccgtaaacaatgacatgattgtgaccgacaacaacggggctgttaagttcccacagctctgtaagttttgcgacgttaggttcagcacctgtgataatcagaagagctgcatgtccaactgcagcatcaccagtatttgcgagaagcctcaagaagtgtgtgtcgctgtttggagaaagaacgacgaaaacataaccctggagaccgtttgccacgatccaaaactcccatatcacgatttcattctggaggacgccgccagtcctaaatgtataatgaaagagaagaagaaaccaggggagaccttctttatgtgcagctgcagcagcgacgagtgtaacgataatataatttttagcgaggagtataatacaagcaatcccgacgtcgagtgccctccatgccctgcccctcctgttgccggacctagtgtgtttttgtttcctcctaaacctaaagatacactcatgattagcaggacacctgaggtgacatgtgtcgtcgtggacgtgagtcatgaagaccccgaagtgcagtttaattggtatgtcgacggagtcgaagtccataatgccaaaactaaaccaagggaagaacagtttaattcaacttttcgcgtggtctctgtgctgactgtggtgcaccaggactggcttaatggaaaggaatacaagtgtaaggtgagtaataagggcctgcccgcccccattgaaaaaactattagtaagactaaagggcagccccgagagccccaggtgtatactttgcccccctctcgggaggagatgactaaaaatcaggtgagtcttacatgtcttgtgaaaggattttacccctctgacatttcagtggagtgggagtctaatggccagcccgagaataattacaaaactactccccccatgttggactctgacggctcatttttcttgtactctaaactgacagtggacaaaagtcggtggcagcagggcaatgtgttttcttgttcagtgatgcacgaggccctgcataatcactatacacagaaatctctgtctctgtcacccggctgatga

SEQ ID NO: 34 - protein, 5 aa, synthetic construct
  REGION 1..5: note = Synthetic
  REPEAT 1..5: note = May be repeated between 1 and 50 times
GGGGS

SEQ ID NO: 35 - protein, 15 aa, synthetic construct
  REGION 1..15: note = Synthetic
ERKCCVECPPCPAPP

SEQ ID NO: 36 - protein, 10 aa, synthetic construct
  REGION 1..10: note = Synthetic
VECPPCPAPP

SEQ ID NO: 37 - protein, 11 aa, synthetic construct
  REGION 1..11: note = Synthetic
THTCPPCPAPE

SEQ ID NO: 38 - protein, 19 aa, synthetic construct
  REGION 1..19: note = Synthetic
VEPKSSDKTHTCPPCPAPE

SEQ ID NO: 39 - protein, 22 aa, synthetic construct
  REGION 1..22: note = Synthetic
GGGSGGGSGGGTHTCPPCPAPE

SEQ ID NO: 40 - protein, 7 aa, synthetic construct
  REGION 1..7: note = Synthetic
PPCPAPE

SEQ ID NO: 41 - protein, 13 aa, synthetic construct
  REGION 1..13: note = Synthetic
DKTHTCPPCPAPE

SEQ ID NO: 42 - protein, 10 aa, synthetic construct
  REGION 1..10: note = Synthetic
VECPPCPAPP

(19) United States
(12) Patent Application Publication
(10) Pub. No.: US 2024/0101641 A1
(43) Pub. Date: Mar. 28, 2024

(54) TGF-B-RECEPTOR ECTODOMAIN FUSION MOLECULES AND USES THEREOF

(71) Applicant: National Research Council of Canada, Ottawa (CA)

(72) Inventors: Anne E.G. LENFERINK, Lorraine (CA); John C. Zwaagstra, Laval (CA); Traian Sulea, Kirkland (CA); Maureen D. O'Connor-McCourt, Beaconsfield (CA)

(73) Assignee: National Research Council of Canada, Ottawa (CA)

(21) Appl. No.: 18/457,588

(22) Filed: Aug. 29, 2023

Related U.S. Application Data
(63) Continuation of application No. 16/490,335, filed on Aug. 30, 2019, now Pat. No. 11,866,481.
(60) Provisional application No. 62/468,586, filed on Mar. 8, 2017.
(60) Provisional application No. 62/465,969, filed on Mar. 2, 2017.

(51) Int. Cl. C07K 14/71; A61P 35/00

(52) U.S. Cl. CPC C07K 14/71; A61P 35/00; A61K 38/00

(57) ABSTRACT

The present invention relates, in general, to polypeptides capable of binding and neutralizing transforming growth factor beta (TGF-β) ligands, and uses of these polypeptides for treating disorders related to TGF-beta expression or activation (e.g. cancer and fibrotic diseases), and methods of making such molecules.

### CROSS-REFERENCE TO RELATED APPLICATIONS

This application is a continuation of U.S. application Ser. No. 16/490,335, filed Aug. 30, 2019, which is a U.S. National Stage Application under 35 U.S.C. § 371 of International Application No. PCT/162018/051320, filed Mar. 1, 2018, which claims priority to U.S. Provisional Application No. 62/465,969, filed Mar. 2, 2017 and U.S. Provisional Application No. 62/468,586, filed Mar. 8, 2017, the entire contents of each of which are hereby incorporated by reference in their entireties.

### REFERENCE TO AN ELECTRONIC SEQUENCE LISTING

The contents of the electronic sequence listing (A089970017US01-SEQ-JRV.xml; Size: 68,478 bytes; and Date of Creation: Aug. 29, 2023) is herein incorporated by reference in its entirety.

### FIELD OF THE INVENTION

The present invention relates to TGF-β receptor ectodomain fusion molecules and uses thereof. More specifically, the present invention relates to TGF-β superfamily receptor ectodomain fusion molecules and their use in TGF-β ligand neutralization.

### BACKGROUND OF THE INVENTION

TGF-β is part of a superfamily of over 30 ligands that regulate several physiological processes, including cell proliferation, migration and differentiation. Perturbation of their levels and/or signaling gives rise to significant pathological effects. For instance, TGF-β and activin ligands play critical pathogenic roles in many diseases including cancer (Hawinkels & Ten Dijke, 2011; Massague et al, 2000; Rodgarkia-Dara et al, 2006). TGF-β, in particular, is considered as a critical regulator of tumor progression and is overexpressed by most tumor types. It favors tumorigenesis in part by inducing an epithelial-mesenchymal transition (EMT) in the epithelial tumor cells, leading to aggressive metastasis (Thiery et al, 2009). TGF-β also promotes tumorigenesis by acting as a powerful suppressor of the immune response in the tumor microenvironment (Li et al, 2006). In fact, TGF-β is recognized as one of the most potent immunosuppressive factors present in the tumor microenvironment. TGF-β interferes with the differentiation, proliferation and survival of many immune cell types, including dendritic cells, macrophages, NK cells, neutrophils, B-cells and T-cells; thus, it modulates both innate and adaptive immunity (Santarpia et al, 2015; Yang et al, 2010). The importance of TGF-β in the tumor microenvironment is highlighted by evidence showing that, in several tumor types (including melanoma, lung, pancreatic, colorectal, hepatic and breast), elevated levels of TGF-β ligand are correlated with disease progression and recurrence, metastasis, and mortality. Hence, significant effort has been invested in devising anti-tumor therapeutic approaches that involve TGF-β inhibition (Arteaga, 2006; Mourskaia et al, 2007; Wojtowicz-Praga, 2003). These approaches include the use of polypeptide fusions based on the TGF-β receptor ectodomain that binds or “traps” the TGF-β ligand (see WO001/83525; WO2005/028517; WO2008/113185; WO2008/157367; WO2010/003118; WO2010/099219; WO2012/071649; WO2012/142515; WO2013/000234; U.S. Pat. No. 5,693,607; US2005/0203022; US2007/0244042; U.S. Pat. Nos. 8,318,135; 8,658,135; 8,815,247; US2015/0225483; and US2015/0056199).

One approach to developing therapeutic agents that inhibit TGF-13 function has been to use antibodies or soluble decoy receptors (also termed receptor ectodomain (ECD)-based ligand traps) to bind and sequester ligand, thereby blocking access of ligand to its cell surface receptors (Zwaagstra et al, 2012). In general, receptor ECD-based traps are a class of therapeutic agents that are able to sequester a wide range of ligands and that can be optimized using protein-engineering approaches (Economides et al, 2003; Holash et al, 2002; Jin et al, 2009).

Previously, a novel protein engineering design strategy was used to generate single-chain, bivalent TGF-β Type II receptor ectodomain (TβRII-ECD) traps that are able to potently neutralize members of the TGF-β superfamily of ligands due to avidity effects (Zwaagstra et al, 2012) [WO 2008/113185; WO 2010/031168]. In this case, bivalency was achieved via covalent linkage of two TβRII ectodomains using regions of the intrinsically disordered regions (IDR) that flank the structured, ligand-binding domain of TβRII-ECD. An example of these single-chain bivalent traps, T22d35, exhibited TGF-β neutralization potencies ˜100-fold higher than the monovalent non-engineered TβRII ectodomain, though the bivalent trap did not neutralize the TGF-β2 isotype and had a relatively short circulating half-life.

It would be useful to provide TβRII-ECD-based traps having improved properties, such as enhanced potency.

### SUMMARY OF THE INVENTION

The present invention provides a polypeptide construct with enhanced potencies in inhibiting TGFβ.

A polypeptide construct of the present invention comprises a first region and a second region, wherein the first region comprises a first and/or second TGFβ receptor ectodomain (ECD); and wherein the second region comprises the second constant domain (C_{H}2) and/or third constant domain (C_{H}3) of an antibody heavy chain. In a preferred non-limiting embodiment, the C-terminus of the first region is linked to the N-terminus of the second region. In a preferred non-limiting embodiment, the first region of the polypeptide construct comprises a first TβRII-ECD (ECD1) and/or a second TβRII-ECD (ECD2), wherein ECD1 and ECD2 are linked in tandem.

The polypeptide construct provided, wherein the first region comprises a (TβRII-ECD)-(TβRII-ECD) doublet linked at its C-terminus with an antibody constant domain inhibits TGFβ activity with at least 600-fold more potency than a counterpart construct having a single TβRII-ECD linked at its C-terminus with an antibody constant domain (i.e when a second ECD is absent, also referred to herein as a singlet).

The polypeptide construct provided comprises a second, TGFβ receptor ectodomain ECD that is linked in tandem to the first ECD, wherein the polypeptide construct (i.e. an ECD doublet construct) linked to an antibody constant domain exhibits TGFβ neutralization (inhibits) that is at least 100, 200, 300, 400, 500, 600, 700, 800 or 900-fold greater than a counterpart construct in which the antibody constant domain is absent, (i.e. an ECD doublet construct, also referred to herein as a non-Fc fused doublet).

In connection with the TβRII-ECDs and the potency with which they inhibit TGF-β activity, it has been found that surprisingly enhanced potencies can result from careful selection of their constituents. This occurs when certain TβRII-ECDs are linked in tandem, and the C-terminus thereof is linked to the N-terminus of an antibody constant domain (Fc). When in their fused and dimeric form, comprising two such polypeptides cross-linked via cysteine bridging between the constant domain/s of each polypeptide, the resulting so-called “Fc fusions” having two TβRII-ECDs (an ECD “doublet”) on each of the two “arms” can exhibit an inhibiting activity that is over 600-fold greater for TGF-β1, and over 20-fold greater for TGF-β3, as compared to “Fc fusions” having one ectodomain on each of the two “arms”, as demonstrated by the inhibition of TGF-β1 and -β3-induced IL-11 secretion by human non-small cell lung cancer (NSCLC) A549 cells, among others. The potency enhancement is evident, relative to counterparts that either lack the Fc region or that lack a second ECD (i.e. are an ECD “singlet”). The potency enhancement is at least 100, 200, 300, 400, 500, or 600-fold greater for the Fc-fused doublet over the Fc-fused singlet. The potency enhancement is at least 100, 200, 300, 400, 500, 600, 700, 800, 900-fold and approximately 1000-fold greater for the Fc-fused doublet over the non-Fc doublet. The potency enhancement is evident, relative to counterparts that either lack the Fc region (the Fc-fused doublet T22d35-Fc is 972- and 243-fold better for TGF-β1 and TGF-β3, respectively than the non-Fc doublet), or that lack a second ECD (an ECD “singlet”; the Fc-fused doublet T22d35-Fc is 615- and 24-fold better for TGF-β1 and TGF-β3, respectively than the non-Fc doublet). More specifically, the Fc-doublet (T22d35-Fc) exhibits a potency enhancement that is at least 970-fold greater for TGF-β1 and at least 240-fold greater for TGF-β3 when compared to a non-Fc fused ECD doublet. Moreover, the Fc-doublet (T22d35-Fc) exhibits a potency enhancement that is at least 600-fold greater for TGF-β1 and over 20-fold greater for TGF-β3 when compared to an Fc-singlet (T2m-Fc).

In a general aspect, there is provided a polypeptide construct comprising at least two TβRII-ECDs linked in tandem (i.e. ECD doublet), and an antibody constant domain comprising at least, the second constant domain (C_{H}2) and/or third constant domain (C_{H}3) of an antibody heavy chain, wherein the C-terminus of the ectodomains is linked to the N-terminus of the antibody constant domain. In this form, the construct is a single chain polypeptide. Thus, the antibody constant domain may comprise only the C_{H}2 domain, or it may comprise the C_{H}2 domain and the C_{H}3 domain.

In embodiments the polypeptides are provided as dimeric fusion polypeptides comprising two single chain polypeptides and cross-linking means coupling the chains covalently.

In other embodiments, the two ectodomains are the same, in terms of their binding targets generally and/or their target species.

In a preferred embodiment, the first region comprises two TGF-β receptor ectodomains (TGFβR-ECD or ‘TβR-ECD’). In a preferred embodiment, the TβR-ECD is a TGF-β receptor type II ectodomain (TβRII-ECD). In a preferred embodiment, the TβR-ECD comprises SEQ ID NO:1, and a sequence substantially identical thereto.

The second region may comprise a sequence selected from the group consisting of SEQ ID NO:12, SEQ ID NO:15, SEQ ID NO:18, SEQ ID NO:24, and a sequence substantially identical thereto.

In a preferred embodiment, the second region of a polypeptide construct of the present invention can further comprise a C_{H}1. In embodiments, the constructs are monofunctional.

The polypeptide constructs of the present invention exploit an antibody heavy chain of human origin. In a preferred embodiment, the antibody heavy chain is selected from the group consisting of a human IgG1 (SEQ ID NO:15) and IgG2 (SEQ ID NO:24).

Thus, in another aspect, there is provided a polypeptide construct according to the present invention wherein the construct is a dimeric polypeptide; wherein the dimeric polypeptide comprises:
(i) a first single chain polypeptide comprising a second region comprising the second constant domain (C_{H}2) and third constant domain (C_{H}3) of an antibody heavy chain, and a first region comprising two TGF-β receptor ectodomains (TβRII-ECD), wherein the C-terminus of the first region is linked to the N-terminus of the second region, and
(ii) a second single chain polypeptide comprising a second region comprising the second constant domain (C_{H}2) and third constant domain (C_{H}3) of an antibody heavy chain, and a first region comprising two TGF-β receptor ectodomains (TβRII-ECD) linked in tandem, wherein the C-terminus of the first region is linked to the N-terminus of the second region, and the first single chain polypeptide is cross-linked with the second single chain polypeptide.

There is also provided a nucleic acid molecule encoding any polypeptide construct of the present invention. There is also provided a vector comprising the nucleic acid molecule of the present invention.

There is also provided a composition comprising one or more than one independently selected polypeptide constructs of the present invention and a pharmaceutically-acceptable carrier, diluent, or excipient.

There is also provided a transgenic cellular host comprising a nucleic acid molecule or a vector of the present invention. The transgenic cellular host can further comprise a second nucleic acid molecule or a second vector encoding a second polypeptide construct when that second polypeptide construct is the same or different from the first polypeptide construct. The second nucleic acid molecule or second vector is present necessarily when the two polypeptide constructs are different (heterodimeric), but are not necessary when the constructs are the same (homodimeric).

There is also provided the use of a polypeptide construct according to the present invention for treatment of a medical condition, disease or disorder; wherein the medical condition, disease or disorder comprises, but is not limited to, cancer, ocular diseases, fibrotic diseases, or genetic disorders of connective tissue and immune disorders.

The polypeptide construct of the present invention may comprise a C_{H}2 and C_{H}3 or only a C_{H}2 from an antibody heavy chain that is of human origin. For example, and without wishing to be limiting, the antibody heavy chain may be selected from the group consisting of a human IgG1 and IgG2. In embodiments, the constant domain in the constructs is C_{H}2 per se, or C_{H}3 per se or C_{H}2-C_{H}3. Suitably, the antibody heavy chain component provides for disulfide crosslinking between single chain polypeptide constructs that are the same or different. Also suitably, the antibody heavy chain provides for protein A-based isolation of the dimeric polypeptide that is produced by the host cells.

In embodiments the receptor ectodomain region comprises two independently selected ectodomains that are linked in tandem, i.e., in a linear manner. In some embodiments, the ectodomains are the same in sequence, or least the same with respect to their target ligand.

The present invention also provides a nucleic acid molecule encoding the polypeptide constructs as described herein. A vector comprising the nucleic acid molecule just described is also encompassed by the invention. The invention also includes a transgenic cellular host comprising the nucleic acid molecule or a vector as described herein; the cellular host may further include a second nucleic acid molecule or a second vector encoding a second polypeptide construct different from the first polypeptide construct. Systems used to produce the present polypeptides can be secretion systems, particularly in the case where dimerization through disulfide bridges is required, and the expression polynucleotides thus encode secretion signals that are cleaved by the host upon secretion into the culturing medium.

Compositions comprising one or more than one independently selected polypeptide construct described herein and a pharmaceutically-acceptable carrier, diluent, or excipient are also encompassed by the present invention.

These and other features of the invention will now be described by way of example, with reference to the appended drawings, wherein:

### BRIEF DESCRIPTION OF THE DRAWINGS

FIG. 1A/B is a schematic drawing of the of the TGF-β Type II receptor ectodomain (TβRII-ECD; also abbreviated as T2m) and the single chain fusion of two T2m domains (also abbreviated T22d35) (FIG. 1A); FIG. 1B provides the corresponding amino acid sequences, wherein the natural linker sequences (SEQ ID NO:6, SEQ ID NO:7, SEQ ID NO:8) are underlined and the sequence of the TβRII structured domain (SEQ ID NO:4) is shown in bold.

FIG. 2A-G, where FIG. 2A/B/C is a schematic representation of the fusion of the T2m and T22d35 modules to the N-termini of the heavy chains of an IgG2 Fc region (2A) in order to generate fusion proteins T2m-Fc (2B) and T22d35-Fc (2C); FIG. 2D provides the amino acid sequence of the T2m-Fc and T22d35-Fc fusion proteins (SEQ ID NO:9, SEQ ID NO:10). FIG. 2E provides aligned sequenced of additional variants of the linker region between the Fc and ECD region in the T22d35-Fc fusions. FIG. 2F and 2G provide the amino acid sequence of the T22d35-Fc linker variants using a human IgG1 Fc (FIG. 2F; SEQ ID NO: 14, SEQ ID NO:17, SEQ ID NO:20) and a human IgG2 Fc region (FIG. 2G; SEQ ID NO:23). The natural linker sequences (SEQ ID NO:6, SEQ ID NO:7, SEQ ID NO:8) are underlined, the sequence of the TβRII structured domain (SEQ ID NO:4) is shown in bold and the human IgG Fc sequence variants (SEQ ID NO:12, SEQ ID NO:15, SEQ ID NO:18, SEQ ID NO:24) are shown in bold-italics.

FIG. 3A/B/C shows the preparative SEC elution profile for the T2m-Fc fusion protein (3A); Fractions (Fr.) 6-11 were pooled and concentrated. Protein integrity of the SEC purified material was then assessed by UPLC-SEC profile (3B) and SDS-PAGE assessment under non-reducing (NR) and reducing (R) conditions (3C).

FIG. 4A/B/C shows the preparative SEC elution profile for the T22d35-Fc fusion protein (4A); Fr. 7-10 were pooled and concentrated. Protein integrity of the SEC purified material was then assessed by UPLC-SEC profile (4B) and SDS-PAGE assessment under non-reducing (NR) and reducing (R) conditions (4C).

FIG. 5A/B provides SDS-PAGE analysis of the protein A purified T22d35-Fc, T22d35-Fc-IgG2-v2(CC), T22d35-Fc-IgG1-v1(CC), T22d35-Fc-IgG1-v2(SCC), T22d35-Fc-IgG1-v3(GSL-CC), hIgG1FeΔK(C)-T22d35, hIgG1FeΔK(CC)-T22d35 and hIgG2FeΔK(CC)-T22d35 variants under non-reducing (A) and reducing (B) conditions.

FIG. 6A/B/C provides the percentage of intact monomer (A), aggregates (B), and fragments (C) of the various fusion proteins, indicating that there are advantages to expressing the T22d35 doublet at the N-terminus of an IgG Fc portion. The table lists the numerical differences in the parameters that were analyzed.

FIG. 7A/B/C provides a functional evaluation of the T2m-Fc and T22d35-Fc fusion proteins compared to the non-Fc-fused single chain T22d35 trap in a A549 IL-11 release assay. Neutralization of TGF-β1 (5A), -β2 (513), -β3 (5C) was assessed and calculated as a % of the TGF-β control (Average of a triplicate experiment+/−SD). The table lists the calculated IC₅₀ values calculated in Graphpad Prism (4-PL algorithm ((log (inhibitor) vs. response—variable slope (four parameters)).

FIG. 8 provides a functional evaluation of the T22d35-Fc, T22d35-Fc-IgG2-v2(CC), T22d35-Fc-IgG1 -v1 (CC), T22d35-Fc-IgG1 -v2(SCC), and T22d35-Fc-IgG1 -v3(GSL-CC) compared to the C-terminal Fc-fused T22d35 trap variants in an A549 IL-11 release assay. Neutralization of TGF-β1, was assessed and calculated as a % of the TGF-β1 control (Average of a triplicate experiment+/−SD). The table lists the calculated IC₅₀ values calculated in Graphpad Prism (4-PL algorithm ((log (inhibitor) vs. response—variable slope (four parameters)).

FIG. 9 provides a functional evaluation of the neutralization TGF-β1, -β2, and β3 by the T22d35-Fc-IgG1-v1(CC) variant in an A549 IL-11 release assay. TGF-β neutralization was assessed and calculated as a % of the TGF-β control (Average of a triplicate experiment+/−SD). The table lists the calculated IC₅₀ values calculated in Graphpad Prism (4-PL algorithm ((log (inhibitor) vs. response—variable slope (four parameters)).

FIG. 10 Functional in vivo evaluation of the T22d35-Fc fusion protein in a syngeneic MC-38 mouse colon carcinoma model. (A) Tumor volumes were calculated as described and plotted as average tumor values+/−SD per cohort. A two-way ANOVA was used to analyse whether statistically significant differences between the calculated average tumor volumes in the T22d35-Fc and CTL IgG treatment cohorts over the course of time. In addition, MC-38 tumor growth (calculated volume) was plotted per individual mouse for the CTL IgG (B) and T22d35-Fc treated cohorts (C).

Additional aspects and advantages of the present invention will be apparent in view of the following description. The detailed descriptions and examples, while indicating preferred embodiments of the invention, are given by way of illustration only, as various changes and modifications within the scope of the invention will become apparent to those skilled in the art

### DETAILED DESCRIPTION OF THE INVENTION

There are now provided polypeptide constructs that bind to and neutralize all transforming growth factor beta (TGF-β1, β2 and β3) isoforms. These polypeptides exploit the TGF-β receptor ectodomains to trap or sequester various TGFβ species including TGF-β1 and TGF-β3, and to some extend TGF-β2. The potency with which the present polypeptide constructs neutralize TGF-β1 and -β3 is surprisingly far greater than related constructs, as demonstrated herein. For this reason, the present constructs are expected to be especially useful as pharmaceuticals for the treatment of medical indications such as cancer, fibrotic diseases and certain immune disorders.

The present polypeptide constructs comprise two TβR-ECDs, such as TβRII-ECDs, that are linked in tandem (C-terminus to N-terminus) and further comprise an antibody constant domain that comprises at least the second constant domain (C_{H}2) and/or third constant domain (C_{H}3) of an antibody heavy chain. The antibody constant domain (Fc) is coupled at its N-terminus to the C-terminus of the ectodomain. Having the ectodomain as a doublet, and having that doublet coupled to the N-terminus of the antibody constant domain, provides a “trap” with an enhanced the neutralization potency by a factor of 615 for TGFβ1 and a factor 24 for TGFβ3 compared to the construct having a single ECD coupled to the N-terminus of the antibody constant domain.

As used herein, the term TβRII-ECD refers to the extracellular region of the TGF-β Type II receptor that binds to the TGF-β ligand. In a preferred embodiment of the present constructs, the TGFβRII ectodomain is the ectodomain of the TGFβR species (i.e. TβRII-ECD) comprising the sequence that forms a stable three-dimensional folded structure:

(SEQ ID NO: 4)
QLCKFCDVRFSTCDNQKSCMSNCSITSICEKPQEVCVAVWRKNDENITLE
TVCHDPKLPYHDFILEDAASPKCIMKEKKKPGETFFMCSCSSDECNDNII
F.

In a related form that comprises flexible natural flanking sequence, the ECD can include the underlined structures, as shown below:

(SEQ ID NO: 1)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPD

This sequence binds to the TGF-β ligand isotypes designated TGF-β1 and TGF-β3. Binding affinity for TGF-β2 is less.

In the present polypeptide constructs, the two TβRII-ECDs may comprise the same sequence. The two ectodomains are linked in tandem, wherein the result is a linear polypeptide in which the C-terminus of one ectodomain is linked to the N-terminus of another ectodomain.

The two ectodomains can be linked by direct fusion such that additional amino acid residues are not introduced. Alternatively, additional amino acid residues can form a linker that couples the two receptor ectodomains in tandem. In the protein construct of the present invention, the first and second regions of the polypeptide construct of the present invention are also linked. By the term “linked”, it is meant that the two regions are covalently bonded. The chemical bond may be achieved by chemical reaction, or may be the product of recombinant expression of the two regions in a single polypeptide chain. In a specific, non-limiting example, the C-terminus of the first region is linked directly to the N-terminus of the second region, that is, no additional “linker” amino acids are present between the two regions. In the case where no linker is present, that is to say direct fusion of the two regions, there will be a direct link between the C-terminus of the full ectodomain and the N-terminus of the antibody constant regions C_{H}2-C_{H}3. For example, in fusing an Fc variant (SEQ ID NO:12, SEQ ID NO:15, SEQ ID NO:18, SEQ ID NO:24) to the SEQ ID NO:1 via the intrinsically disordered linker with SEQ ID NO:8, which is part of the TβRII-ECD having SEQ ID NO:1 (i.e., no additional “linker” amino acids added), one connects the aspartic acid at the last position of SEQ ID NO:1 to a glutamic acid, a threonine, a valine or a valine found at the first position of SEQ ID NO:12, SEQ ID NO:15, SEQ ID NO:18, or SEQ ID NO:24, respectively.

A common practice when producing fusion constructs is to introduce glycine or glycine-serine linkers (GSL) such as GGGGS (SEQ ID NO: 34) or [G4S]n (where n is 1, 2, 3, 4 or 5 or more, such as 10, 25 or 50)(SEQ ID NO: 34) between the fused components. As taught in the above paragraph, the polypeptide fusions of the present invention can be produced by direct linkage without use of any additional amino-acid sequence except those present in the

Fc region and in the receptor ectodomain region. One thus can refrain from utilizing foreign sequences as linkers, providing an advantage due to their potential for undesired immunogenicity and their added molecular weight. Entropic factors are also a potential liability for glycine and glycine-serine linkers, which are highly flexible and may become partially restricted upon target binding, hence causing a loss of entropy unfavourable to binding affinity. Therefore, only the flexible, intrinsically disordered N-terminal regions of the TGFβRII-ECD were employed as natural linkers in embodiments of the present invention. However, the particular amino acid compositions and lengths of these intrinsically disordered linkers (e.g., SEQ ID NO:6, SEQ ID NO:7, SEQ ID NO:8) precluded accurate prediction of whether the resulting direct-fusion constructs will have the required geometry and favourable molecular interactions for correct binding to their intended dimeric ligands. In other embodiments, the fusion polypeptide may include a flexible artificial GSL, as exemplified in the construct in SEQ ID NO:20, where the GS linker with the SEQ ID NO:21 is introduced between the aspartic acid (D) at the last position of TβRII-ECD having SEQ ID NO:1 and the threonine (T) at the first position of the Fc region variant having SEQ ID NO:15.

The first and second regions of the polypeptide construct are, in embodiments, connected by natural intrinsically disordered polypeptide linkers selected from the group consisting of SEQ ID NO:8, 13, 16, 19, 25, and a sequence substantially identical thereto. In other embodiments, the regions of the polypeptide constructs are connected by flexible linkers selected from the group consisting of SEQ ID NO:21 and SEQ ID NO:22, and a sequence substantially identical thereto.

In this embodiment, one region of the present polypeptide constructs comprises a TβRII-ECD doublet comprising first and second receptor ectodomains linked in tandem by the natural intrinsically disordered polypeptide linker with SEQ ID NO: 6, and having amino acid sequence comprising:

(SEQ ID NO: 5)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPDIPPHVQKSVNNDMI
VTDNNGAVKFPQLCKFCDVRFSTCDNQKSCMSNCSITSICEKPQEVCVAV
WRKNDENITLETVCHDPKLPYHDFILEDAASPKCIMKEKKKPGETFFMCS
CSSDECNDNIIFSEEYNTSNPD.

The present constructs also comprise a region that comprises an antibody constant domain that comprises at least the second constant domain (C_{H}2) and/or third constant domain (C_{H}3) of an antibody heavy chain. The antibody constant domain is coupled at its N-terminus to the C-terminus of the ectodomain doublet, so that the orientation of the construct is a single chain of TβRII-ECD(link)TβRII-ECD(link)C_{H}2-C_{H}3.

The antibody constant domain provides for cross-linking between two of the present polypeptide constructs. This is achieved when the expressed polypeptide constructs are secreted from their expression host. Thus, production of the single chain polypeptide provides the construct in a dimeric form in which the two constructs are cross-linked via disulfide bridges that involve one or more cysteine residues within each of the antibody constant domains present in each of the constructs.

The antibody constant domain present in the construct is desirably sourced from an IgG constant region, and especially from the constant domain of either IgG1 or IgG2.

The constructs provided are monofunctional in the sense that the constant region itself may have no particular activity, other than to act as a structure through which dimers of the polypeptide constructs can form. These minimal constant regions can also be altered to provide some benefit, by incorporating the corresponding hinge regions and optionally changing the cysteine residue composition. Thus, some or all of the cysteine residues involved in bridging the two Fc fragments or naturally used to bridge between the heavy and light chains of a full-length antibody can be replaced or deleted. One advantage of minimizing the number of cysteine residues is to reduce the propensity for disulphide bond scrambling, which could promote aggregation. For example, these cysteine residues and alteration thereof are seen in the natural or non-natural linker sequences located around the junction of the first and second regions of the polypeptide constructs and which are listed below:

SEEYNTSNPDTHTCPPCPAPE (SEQ ID NO:16), SEEYNTSNPDVEPKSSDKTHTCPPCPAPE (SEQ ID NO:19), SEEYNTSNPDGGGSGGGSGGGTHTCPPCPAPE (SEQ ID NO:22) incorporating variations of human IgG1 hinge sequence; and SEEYNTSNPDERKCCVECPPCPAPP (SEQ ID NO:13) and SEEYNTSNPDVECPPCPAPP (SEQ ID NO:25) incorporating variations of human IgG2 hinge sequence; and a sequence substantially identical thereto.

Not all of the naturally-occurring inter-hinge disulfide bonds need to be formed for the Fc homodimerization to occur, while noting that the stability of the Fc homodimer may depend on the number of intermolecular disulphide bridges.

In the present disclosure, an “antibody”, also referred to in the art as “immunoglobulin” (Ig), refers to a protein constructed from paired heavy and light polypeptide chains. The structure of an antibody and of each of the domains is well established and familiar to those of skill in the art, though is summarized herein. When an antibody is correctly folded, each chain folds into a number of distinct globular domains joined by more linear polypeptide sequences; the immunoglobulin light chain folds into a variable (V_{L}) and a constant (C_{L}) domain, while the heavy chain folds into a variable (V_{H}) and three constant (C_{H}1, C_{H}2, C_{H}3) domains. Once paired, interaction of the heavy and light chain variable domains (V_{H} and V_{L}) and first constant domain (C_{L} and C_{H}1) results in the formation of a Fab (Fragment, antigen-binding) containing the binding region (Fv); interaction of two heavy chains results in pairing of C_{H}2 and C_{H}3 domains, leading to the formation of a Fc (Fragment, crystallisable). Characteristics described herein for the C_{H}2 and C_{H}3 domains also apply to the Fc.

In the present invention and its specific embodiments, the polypeptide constructs that exhibit significantly enhanced potency comprise the following:

T22d35-Fc:
(SEQ ID NO: 10)
IPPHVQKSVNNDMIVTDNNGAVKFPQLCKFCDVRFSTCDNQKSCMSNCSITSICEKPQEVCVA
VWRKNDENITLETVCHDPKLPYHDFILEDAASPKCIMKEKKKPGETFFMCSCSSDECNDNIIFS
EEYNTSNPDIPPHVQKSVNNDMIVTDNNGAVKFPQLCKFCDVRFSTCDNQKSCMSNCSITSIC
EKPQEVCVAVWRKNDENITLETVCHDPKLPYHDFILEDAASPKCIMKEKKKPGETFFMCSCSS
DECNDNIIFSEEYNTSNPDERKCCVECPPCPAPPVAGPSVFLFPPKPKDTLMISRTPEVTCVV
VDVSHEDPEVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDISVEWESNGQP
ENNYKTTPPMLDSDGSFFLYSKLTVDKSRWQQGNVFSCSVMHEALHNHYTQKSLSLSPG
T22d35-Fc-IgG2-v2 (CC):
(SEQ ID NO: 23)
IPPHVQKSVNNDMIVTDNNGAVKFPQLCKFCDVRFSTCDNQKSCMSNCSITSICEKPQEVCVA
VWRKNDENITLETVCHDPKLPYHDFILEDAASPKCIMKEKKKPGETFFMCSCSSDECNDNIIFS
EEYNTSNPDIPPHVQKSVNNDMIVTDNNGAVKFPQLCKFCDVRFSTCDNQKSCMSNCSITSIC
EKPQEVCVAVWRKNDENITLETVCHDPKLPYHDFILEDAASPKCIMKEKKKPGETFFMCSCSS
DECNDNIIFSEEYNTSNPDVECPPCPAPPVAGPSVFLFPPKPKDTLMISRTPEVTCVVVDVSH
EDPEVQFNWYVDGVEVHNAKTKPREEQFNSTFRVVSVLTVVHQDWLNGKEYKCKVSNKGLP
APIEKTISKTKGQPREPQVYTLPPSREEMTKNQVSLTCLVKGFYPSDISVEWESNGQPENNYK
TTPPMLDSDGSFFLYSKLTVDKSRWQQGNVFSCSVMHEALHNHYTQKSLSLSPG
T22d35-Fc-IgG1-v1 (CC):
(SEQ ID NO: 14)
IPPHVQKSVNNDMIVTDNNGAVKFPQLCKFCDVRFSTCDNQKSCMSNCSITSICEKPQEVCVA
VWRKNDENITLETVCHDPKLPYHDFILEDAASPKCIMKEKKKPGETFFMCSCSSDECNDNIIFS
EEYNTSNPDIPPHVQKSVNNDMIVTDNNGAVKFPQLCKFCDVRFSTCDNQKSCMSNCSITSIC
EKPQEVCVAVWRKNDENITLETVCHDPKLPYHDFILEDAASPKCIMKEKKKPGETFFMCSCSS
DECNDNIIFSEEYNTSNPDTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKAL
PAPIEKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPG
T22d35-Fc-IgG1-v2 (SCC):
(SEQ ID NO: 17)
IPPHVQKSVNNDMIVTDNNGAVKFPQLCKFCDVRFSTCDNQKSCMSNCSITSICEKPQEVCVA
VWRKNDENITLETVCHDPKLPYHDFILEDAASPKCIMKEKKKPGETFFMCSCSSDECNDNIIFS
EEYNTSNPDIPPHVQKSVNNDMIVTDNNGAVKFPQLCKFCDVRFSTCDNQKSCMSNCSITSIC
EKPQEVCVAVWRKNDENITLETVCHDPKLPYHDFILEDAASPKCIMKEKKKPGETFFMCSCSS
DECNDNIIFSEEYNTSNPDVEPKSSDKTHTCPPCPAPELLGGPSVFLFPPKPKDTLMISRTPEV
TCVVVDVSHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQKSLSLS
PG;
and
T22d35-Fc-IgG1-v3 (GSL-CC):
(SEQ ID NO: 20)
IPPHVQKSVNNDMIVTDNNGAVKFPQLCKFCDVRFSTCDNQKSCMSNCSITSICEKPQEVCVA
VWRKNDENITLETVCHDPKLPYHDFILEDAASPKCIMKEKKKPGETFFMCSCSSDECNDNIIFS
EEYNTSNPDIPPHVQKSVNNDMIVTDNNGAVKFPQLCKFCDVRFSTCDNQKSCMSNCSITSIC
EKPQEVCVAVWRKNDENITLETVCHDPKLPYHDFILEDAASPKCIMKEKKKPGETFFMCSCSS
DECNDNIIFSEEYNTSNPDGGGSGGGSGGGTHTCPPCPAPELLGGPSVFLFPPKPKDTLMIS
RIPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAV
EWESNGQPENNYKTIPPVLDSDGSFFLYSKLTVDKSRWQQGNVFSCSVMHEALHNHYTQK
SLSLSPG

In a specific embodiment, the polypeptide construct comprises a polypeptide of the present invention that exhibits significantly enhanced potency, for example a polypeptide the exhibits significant enhanced potency may comprise SEQ ID NO: 10, SEQ ID NO.14, SEQ ID NO:17, SEQ ID NO: 20 or SEQ ID NO:23. In another specific embodiment, the polypeptide construct may be a homodimer comprising two polypeptides that exhibit significant enhanced potency; for example if the polypeptide construct that exhibits significant enhanced potency is SEQ ID NO: 14, the polypeptide construct is a homodimer comprising two polypeptide constructs wherein each polypeptide construct comprising SEQ ID NO.14. Likewise, if the polypeptide that exhibits enhanced potency is SEQ ID NO: 10, 14, 17, 20, or 23, a homodimer of the present invention likewise comprises two polypeptide constructs wherein each polypeptide of the homodimer is SEQ ID NO: 10, 14, 17, 20 or 23 respectively.

As noted, these single chain polypeptide constructs will dimerize when secreted from a production host, yielding a dimeric polypeptide construct comprising two single chain polypeptides linked by way of disulfide bridges that form between the constant domains of the two single chain polypeptides.

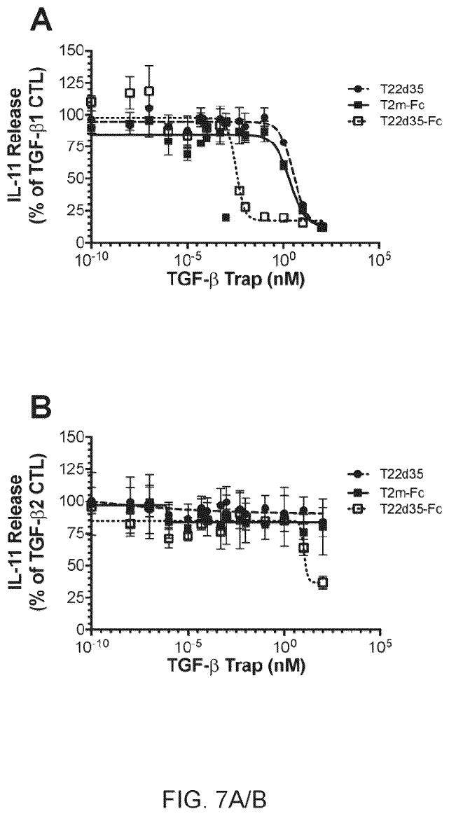

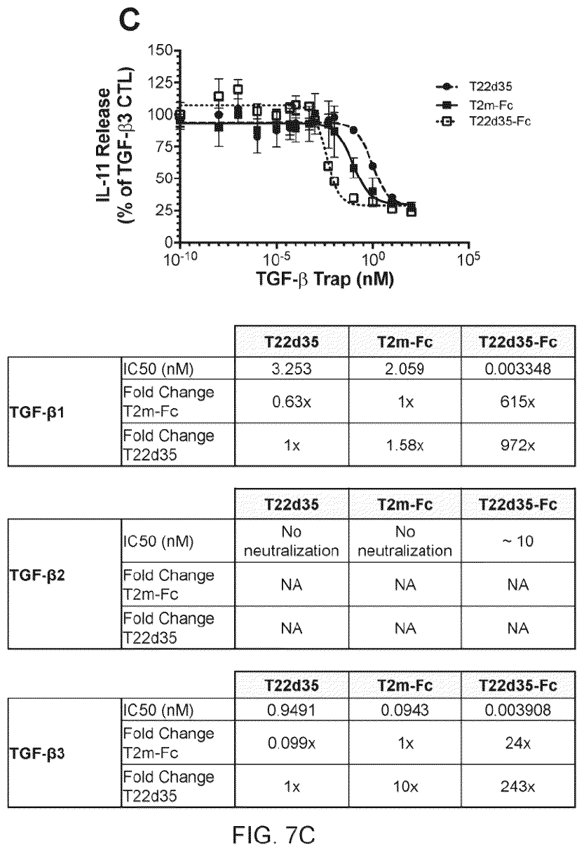

By “significantly enhanced potency” we mean that the effect or activity of a present polypeptide construct in this dimeric form is greater than a counterpart construct when measured in an assay relevant for assessing the biological activity of TGF-β. Appropriate means for making this determination are exemplified herein. For example, the N-terminal Fc-fused T22d35 doublets neutralizes TGF-β to a much better extend than the N-terminally Fc-fused T2m singlet as was illustrated by the TGF-β-induced IL-11 release by A549 cells (FIG. 7).

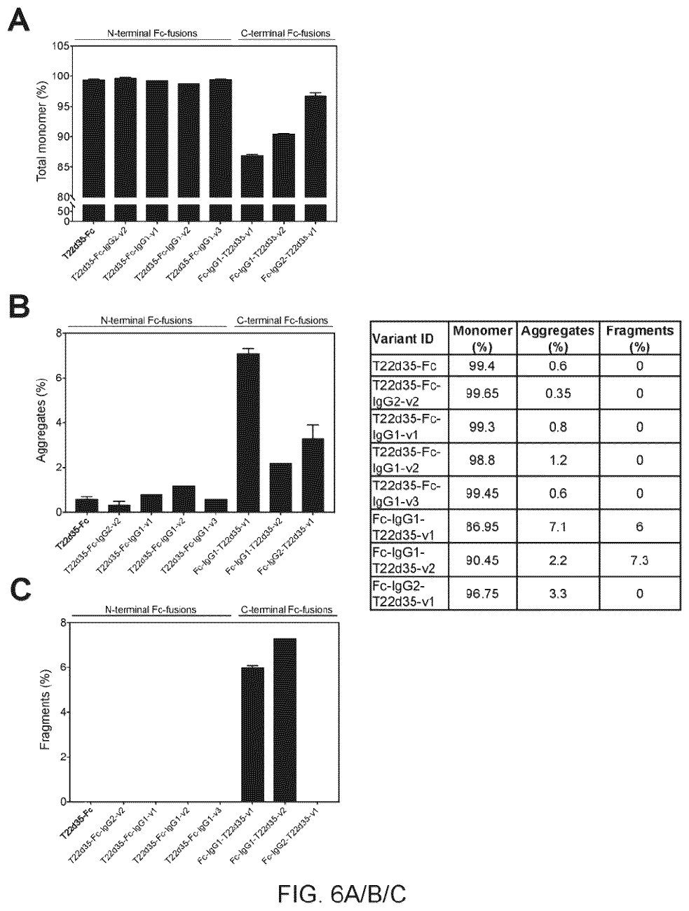

It is observed that fusion constructs of this type have advantages relative to several other versions of TβRII receptor-ectodomain based molecules, including non-Fc fused bivalent TGF-β receptor ectodomain constructs (such as the T22d35 doublet) and constructs in which a single receptor ectodomain is fused to the N-terminus of an Fc region. In particular, the presently provided Fc fusion constructs have improved manufacturability due to the presence of the Fc region (for example, purification can be accomplished using protein A chromatography). The Fc region also allows for improved circulating half-lives. Importantly, the present constructs have substantially higher TGF-β neutralization potencies compared to the singlet fusion (T2m-Fc) and non-Fc-fused doublet ectodomain (T22d35). The N-terminally fused TGF-β ECD doublet Fc constructs (T22d35-Fc) provided exhibit advantages with respect to significant improvement in TGF-β ligand neutralizing potency (as shown, for example, in the over 970-fold improvement in TGF-β1 neutralization relative to non-Fc fused doublet, as shown in FIG. 7). Additionally, they exhibit improved manufacturability, as demonstrated by biophysical analysis showing a >99% monomeric content (i.e. the minimal presence of aggregates and the absence of fragments of the purified N-terminally fused T22d35-Fc constructs) (as illustrated in FIG. 6). Thus, an advantage of the present invention is an unexpected high potency of TGF-β ligand neutralization, including some degree of neutralization of TGF-β2, which is not observed with the T2m-Fc (Fc-singlet) or T22d35 (non Fc-fused) constructs.

In specific embodiments, the second region of the polypeptide construct of the present invention is selected from a group of sequences displaying variation in the N-terminal sequence as exemplified by SEQ ID NO:12, 15, 18, 24. These may differ in length and the number of cysteine residues retained from the hinge region as a means to modulating the degree of Fc-region dimerization and hence impacting on both efficacy and manufacturability. Thus, in embodiments, the polypeptide construct comprises a variation in the constant domain, wherein at least one cysteine residue involved in cross-linking is deleted or substituted. Suitable substitutions include serine or alanine, and preferably by serine.

A substantially identical sequence may comprise one or more conservative amino acid mutations that still provide for proper folding upon secretion into the culturing medium. It is known in the art that one or more conservative amino acid mutations to a reference sequence may yield a mutant peptide with no substantial change in physiological, chemical, physico-chemical or functional properties compared to the reference sequence; in such a case, the reference and mutant sequences would be considered “substantially identical” polypeptides. A conservative amino acid substitution is defined herein as the substitution of an amino acid residue for another amino acid residue with similar chemical properties (e.g. size, charge, or polarity). These conservative amino acid mutations may be made to the framework regions while maintaining the overall structure of the constant domains; thus the function of the Fc is maintained.

In a specific, non-limiting example, the first region of the polypeptide construct of the present invention may comprise a TGF-β receptor type II, such as:

(SEQ ID NO: 1, also referred to herein as T2m)
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIFSEEYNTSNPD.

In preferred embodiments, the polypeptide constructs comprise a TβRII-ECD “doublet”, in which a TβRII-ECD is linked in tandem with another TβRII-ECD, which ectodomains can be the same or different TGF-β superfamily receptor ectodomains, such as:

(SEQ ID NO: 5,
IPPHVQKSVNNDMIVTDNNGAVKFPQLCKFCDVRFSTCDNQKSCMSNCSI
TSICEKPQEVCVAVWRKNDENITLETVCHDPKLPYHDFILEDAASPKCIM
KEKKKPGETFFMCSCSSDECNDNIIF
-linker-
QLCKFCDVRFSTCDNQKSCMSNCSITSICEKPQEVCVAVWRKNDENITLE
TVCHDPKLPYHDFILEDAASPKCIMKEKKKPGETFFMCSCSSDECNDNII
FSEEYNTSNPD

also referred to herein as T22d35) where in one non-limiting embodiment the linker corresponds to SEQ ID NO:6; and
a sequence substantially identical thereto. “Substantially identical” is as defined above.

The ectodomain doublet can incorporate the same or different ectodomains, both belonging to the TGFβ superfamily receptor family. In embodiments, the ectodomains bind the same target. In other embodiments, the ectodomains are of the same receptor species. In other embodiments, the ectodomains are identical and thus are homomeric.

For example, the polypeptide constructs of the present invention may have a TGF-β neutralization potency selected from the group consisting of at least 900-fold and 200-fold, more potent than the T22d35 doublet alone for TGFβ1 and TGF-β3, respectively. For example, in the IL-11 release assay the T22d35-Fc doublet construct is approximately 972-fold more potent in neutralizing TGF-β1 and approximately 243-fold more potent in neutralizing TGF-β3, when compared with the non-Fc-fused T22d35 doublet alone.

In another example, the potency of the construct is at least 600-fold and at least 20-fold greater for neutralizing TGF-β1 and TGF-β3, respectively, than a construct in which the antibody constant domain is coupled to a single ectodomain rather than to a doublet. The polypeptide constructs of the present invention may have an at least 615-fold and 24-fold better neutralization potency for TGF-β1 and TGF-β3, respectively, when compared to the potency of a construct in which the antibody constant domain is coupled to a single ectodomain rather than to a doublet.

The neutralizing potency can be summarized as follows: the neutralizing potency of the Fc-fused doublet (ECD-ECD-Fc) is greater than the Fc-fused ECD monomer (ECD-Fc); i.e. ECD-ECD-Fc>ECD-Fc, whereas the ECD-Fc is more potent than the non-Fc-fused doublet

(ECD-ECD) and the non-Fc fused doublet ECD is more potent than the non-Fc fused singlet ECD; i.e. ECD-ECD-Fc>>ECD-Fc>ECD-ECD>>ECD). In terms of manufacturability, the presence of an Fc protein allows for Protein A purification and prevents having to use cleavable tags. In addition, positioning the singlet or doublet ECD at the N-terminus of the Fc portion prevents aggregation issues due to the inappropriate pairing of the cysteine residues in the hinge region of the Fc portion. Therefore, fusion of the ECD singlet or doublet to the N-terminus of the Fc portion provides an improved manufacturability over C-terminal fusions (N-terminal fusions have a higher percentage of monomeric species, less aggregates, less fragments). In addition an unexpected significant increase is observed in TGF-β neutralization potency for all TGF-β isotypes for the N-terminal Fc fused doublet ECD compared to the N-terminal Fc fused T2m singlet ECD.

Additionally, when the polypeptide constructs of the present invention include a TβRII-ECD that binds TGF-β, the polypeptide construct may neutralize, to varying extents, all three isotypes of TGF-β (that is, TGF-β1, TGF-β2, and TGF-β3).

The polypeptide constructs of the present invention have, as assessed in cell-based assays, TGF-β neutralizing potencies that are significantly higher (20-fold or more) than those of bivalent comparator polypeptides, i.e. non-Fc-fused T22d35 (doublet alone) and T2m-Fc (Fc fused singlet). Within the series of polypeptide constructs of the present invention, those that contain two or more copies of the TβRII-ECD fused to the N-terminus of the Fc constant region have potencies that are higher than those constructs that contain only one copy, as assessed in cell based assays.

The polypeptide construct of the present invention is expressed as a single polypeptide chain. Once expressed, the polypeptide construct of the present invention forms a dimer wherein the C_{H}2 and C_{H}3 domains of the respective polypeptide constructs interact to form a properly assembled Fc region such as occurs when the expressed products are secreted into the culturing medium.

The polypeptide construct of the present invention may also comprise additional sequences to aid in expression, detection or purification of a recombinant antibody or fragment thereof. Any such sequences or tags known to those of skill in the art may be used. For example, and without wishing to be limiting, the antibody or fragment thereof may comprise a targeting or signal sequence (for example, but not limited to ompA), a detection/purification tag (for example, but not limited to c-Myc, His₅, His₆, or His₈G), or a combination thereof. In another example, the signal peptide may be MDWTWRILFLVAAATGTHA (SEQ ID NO:11). In a further example, the additional sequence may be a biotin recognition site such as that described in [WO/1995/04069] or in [WO/2004/076670]. As is also known to those of skill in the art, linker sequences may be used in conjunction with the additional sequences or tags, or may serve as a detection/purification tag. Suitably, the constant region comprises a protein A binding site (residing typically between about C_{H}2 and C_{H}3) that permits the single chain polypeptide to be extracted/isolated using a protein A affinity approach.

The present invention also encompasses nucleic acid sequences encoding the molecules as just described above. Given the degeneracy of the genetic code, a number of nucleotide sequences would have the effect of encoding the desired polypeptide, as would be readily understood by a skilled artisan. The nucleic acid sequence may be codon-optimized for expression in various micro-organisms. The present invention also encompasses vectors comprising the nucleic acids as just described, wherein the vectors typically comprise a promoter and signal sequence that are operably linked to the construct-encoding polynucleotide for driving expression thereof in the selected cellular production host. The vectors can be the same or different provided both result in secretion of the dimeric polypeptide construct.

Furthermore, the invention encompasses cells, also referred to herein as transgenic cellular host, comprising the nucleic acid and/or vector as described, encoding a first polypeptide construct. The host cells may comprise a second nucleic acid and/or vector encoding a second polypeptide construct different from the first polypeptide construct. The co-expression of the first and second polypeptide constructs may lead to the formation of heterodimers.

The present invention also encompasses a composition comprising one or more than one polypeptide construct as described herein. The composition may comprise a single polypeptide construct as described above, or may be a mixture of polypeptide constructs. The composition may also comprise one or more than one polypeptide construct of the present invention linked to one or more than one cargo molecule. For example, and without wishing to be limiting in any manner, the composition may comprise one or more than one polypeptide construct of the present invention linked to a cytotoxic drug in order to generate an antibody-drug conjugate (ADC) in accordance with the present invention.

The composition may also comprise a pharmaceutically acceptable diluent, excipient, or carrier. The diluent, excipient, or carrier may be any suitable diluent, excipient, or carrier known in the art, and must be compatible with other ingredients in the composition, with the method of delivery of the composition, and is not deleterious to the recipient of the composition. The composition may be in any suitable form; for example, the composition may be provided in suspension form, powder form (for example, but limited to lyophilised or encapsulated), capsule or tablet form. For example, and without wishing to be limiting, when the composition is provided in suspension form, the carrier may comprise water, saline, a suitable buffer, or additives to improve solubility and/or stability; reconstitution to produce the suspension is effected in a buffer at a suitable pH to ensure the viability of the antibody or fragment thereof. Dry powders may also include additives to improve stability and/or carriers to increase bulk/volume; for example, and without wishing to be limiting, the dry powder composition may comprise sucrose or trehalose. In a specific, non-limiting example, the composition may be so formulated as to deliver the antibody or fragment thereof to the gastrointestinal tract of the subject. Thus, the composition may comprise encapsulation, time-release, or other suitable technologies for delivery of the antibody or fragment thereof. It would be within the competency of a person of skill in the art to prepare suitable compositions comprising the present compounds.

The constructs of the present invention may be used to treat diseases or disorders associated with over-expression or over-activation of ligands of the TGF-β superfamily. The disease or disorder can be selected from, but not limited to, cancer, ocular diseases, fibrotic diseases, or genetic disorders of connective tissue.

In the field of cancer therapy, it has recently been demonstrated that TGF-β is a key factor inhibiting the antitumor response elicited by immunotherapies, such as immune checkpoint inhibitors (ICI's) (Hahn & Akporiaye, 2006). Specifically, therapeutic response to ICI antibodies results primarily from the re-activation of tumor-localized T-cells. Resistance to ICI antibodies is attributed to the presence of immunosuppressive mechanisms that result in a dearth of T-cells in the tumor microenvironment. Thus, it is now recognized that in order to elicit responses in resistant patients, ICI antibodies need to be combined with agents that can activate T-cells and induce their recruitment into the tumor, i.e. reversing of the “non-T-cell-inflamed” tumor phenotype. One publication noted that overcoming the non-T-cell-inflamed tumor microenvironment is the most significant next hurdle in immuno-oncology (Gajewski, 2015).

We have shown using a proof-of-principle TGF-β trap, T22d35, that blocking of TGF-β effectively reverses the “non-T cell inflamed” tumor phenotype (Zwaagstra et al, 2012). This positions anti-TGF-β molecules as potential synergistic combinations with ICI's and other immunotherapeutics. In support of this, a 2014 study (Holtzhausen et al., ASCO poster presentation) examined effects of a TGF-β blocker when combined an anti-CTLA-4 antibody in a physiologically-relevant transgenic melanoma model. The study demonstrated that while anti-CTLA-4 antibody monotherapy failed to suppress melanoma progression, the combination of the TGF-β antagonist and anti-CTLA-4 antibody significantly and synergistically suppressed both primary melanoma tumor growth as well as melanoma metastasis. These observations correlated with significant increases in effector T-cells in melanoma tissues.

We show herein that the present polypeptides having the basic structure that is T22d35-Fc significantly reduce tumor growth in a syngeneic mouse MC-38 colon cancer model. This thus positions anti-TGF-β molecules to be used in a potential synergistic combination with other immunotherapeutics.

The present constructs can be useful to treat fibrotic diseases including those that affect any organ of the body, including, but not limited to kidney, lung, liver, heart, skin and eye. These diseases include, but are not limited to, chronic obstructive pulmonary disease (COPD), glomerulonephritis, liver fibrosis, post-infarction cardiac fibrosis, restenosis, systemic sclerosis, ocular surgery-induced fibrosis, and scarring.

Genetic disorders of connective tissue can also be treated, and include, but are not limited to, Marfan syndrome (MFS) and Osteogenesis imperfecta (OI).

The present invention will be further illustrated in the following examples. However, it is to be understood that these examples are for illustrative purposes only and should not be used to limit the scope of the present invention in any manner.

### Materials & Methods

### Production & Purification

### Transient CHO Expression

The various TβRII-ECD fusion variants (such as T2m-Fc and T22d35-Fc) are each comprised of a heavy chain Fc region, and include the signal sequence MDWTWRILFLVAAATGTHA (SEQ ID NO:11) at their N-termini. The DNA coding regions for the constructs were prepared synthetically (Biobasic Inc. or Genescript USA Inc.) and were cloned into the HindIII (5′ end) and BamH1 (3′ end) sites of the pTT5 mammalian expression plasmid vector (Durocher et al, 2002). Fusion proteins were produced by transient transfection of Chinese Hamster Ovary (CHO) cells with the heavy chain T2m or T22d35 fused to the IgG heavy chain (T2m-Hc and T22d35-HC, respectively) construct. Briefly, T2m-HC or T22d35-HC plasmid DNAs were transfected into a 2.5 L and 4.6 L culture, respectively, of CHO-3E7 cells in FreeStyle F17 medium (Invitrogen) containing 4 mM glutamine and 0.1% Kolliphor p-188 (Sigma) and maintained at 37° C. Transfection conditions were: DNA (80% plasmid construct, 15% AKT plasmid, 5% GFP plasmid): PEIpro (ratio 1:2.5): PEI(polyethylenimine)pro (Polyplus) (ratio=1:2.5). At 24 hours post-transfection, 10% Tryptone N1 feed (TekniScience Inc.) and 0.5 mM Vaporic acid (VPA, Sigma) were added and the temperature was shifted to 32° C. to promote the production and secretion of the fusion proteins and then maintained for 15 days post transfection after which the cells were harvested. At final harvest the cell viability was 89.6%.

### Stable Pool CHO Expression

CHO^{BRI/rcTA} cell pools were generated by transfecting cells with the vector expressing the target gene encoding the various Fc-fused TβRII-ECD proteins. The day after transfection, the cells were centrifuged for 5 min at 250 rpm and seeded at density of 0.5×10⁶ cells/mL in selection medium (PowerCHO2 medium supplemented with 50 μM of methionine sulfoximine). Selection medium was replaced every 2-3 days during 14 to 18 days with inoculation at 0.5×10⁶ cells/mL. Cell number and viability were measured with the Cedex Innovatis' automated cell counter Cedex Analyzer as described above. When cell viability reached greater than 95%, pools were inoculated at 0.2×10⁶ cells/mL in 125 or 250 mL Erlenmeyer flasks. For the fed-batch culture, CHO^{BRI/rcTA} cell pools were inoculated as described above. At day three post-inoculation, when cells density reached 3.5 to 4.5×10⁶ cells/mL, expression of the recombinant protein was induced by adding 2 μg/mL of cumate. MSX concentration was adjusted to 125 μM, and F12.7 feed (Irvine Scientific) was added followed by a temperature shift to 32° C. Every 2-3 days, cultures were fed with 5% (v:v) F12.7 and samples were collected for recombinant protein (pA-HPLC) and glucose (VITROS 350, Orthoclinical Diagnostics, USA) concentration determination. Glucose was added in order to maintain a minimal concentration of 17 mM.

### Purification

The harvest supernatant from the CHO cells was filtered (0.2 μm) and loaded onto a Protein A MabSelect Sure column (GE Healthcare). The column was washed with 2 column volumes of PBS and protein was eluted with 3 column volumes of 0.1 M sodium citrate pH 3.6. To maximize the yield, the flow through was reloaded onto the Protein A column and eluted as described above. Eluted fractions were neutralized with 1 M Tris, and those containing the fusion proteins were pooled and subsequently loaded onto a Hi-load Superdex S200 26/60 size exclusion chromatography (SEC) column (GE Healthcare) equilibrated in formulation buffer (DPBS without Ca²⁺, without Mg²⁺). Protein was eluted using 1 column volume formulation buffer, collected into successive fractions and detected by UV absorbance at 280 nm. The main peak SEC fractions containing the fusion proteins were then pooled and concentrated. The integrity of the Prot-A and SEC purified fusion proteins in the pooled fractions was further analyzed by UPLC-SEC and SDS-PAGE (4-15% polyacrylamide) under reducing and non-reducing conditions (SYPRO Ruby staining). For UPLC-SEC, 2-10 μg of protein in DPBS (Hyclone, minus Ca²⁺, minus Mg²⁺) was injected onto a Waters BEH200 SEC column (1.7 μm, 4.6×150 mm) and resolved under a flow rate of 0.4 mL/min for 8.5 min at room temperature, using the Waters Acquity UPLC H-Class Bio-System. Protein peaks were detected at 280 nm (Acquity PDA detector).

### Cell Lines

Human A549 non-small cell lung cancer cells were purchased from ATCC (Cat #CCL-185, Cedarlane, Burlington, ON). Cells were cultured in Dulbecco's Modified Eagles Medium (DMEM) supplemented with 5% Fetal Bovine Serum (FBS). MC-38 mouse colon adenocarcinoma cells were purchased from Kerafast (Cat #ENH204, Boston, MA), and cultured in Dulbecco's modified MEM supplemented with 2 mM L-glutamine and 10% fetal bovine serum. Both cell lines were maintained at 37° C., in a humidified atmosphere supplemented with 5% CO₂.

### TGF-β Induced A549 Cell IL-11 Release Assay

Human A549 lung cancer cells were seeded in 96-well plates (5×10³ cells/well). The following day 10 pM TGF-β in complete media, in the absence or presence of a serial dilution of TGF-β Trap fusion protein, was incubated for 30 min at RT prior to adding to the cells. After 21 h of incubation (37° C., 5% CO₂, humidified atmosphere) conditioned medium was harvested and added to MSD Streptavidin Gold plates (Meso Scale Diagnostics, Gaithersburg, MD) that were coated with 2 μg/mL biotinylated mouse anti-human IL-11 antibody (MAB618, R&D Systems, Minneapolis, MN). After 18 h (4° C.) plates were washed with PBS containing 0.02% Tween 20 and then 2 μg/mL SULFO-tagged goat anti-human IL-11 antibody (AF-218-NA, R&D Systems Minneapolis, MN) was added and plates were incubated for 1 h at RT. After a final wash, plates were read in a MESO QuickPlex SQ120 machine (Meso Scale Diagnostics, Gaithersburg, MD). IL-11 readouts were expressed as percent IL-11 release compared to control cells treated with TGF-β alone. Graphpad Prism (4-PL algorithm ((log (inhibitor) vs. response—variable slope (four parameters)) was used to calculate the IC₅₀ (the automatic outlier option was used when needed).

### In vivo Evaluation in a Syngeneic Mouse Colon Cancer MC-38 Subcutaneous Mouse Model

Female C57BL/6-Elite mice (5-7 weeks old) were purchased from Charles River Laboratories (Wilmington, MA). Thirteen C57BL/6 mice were injected on day 0 with 3×10⁵ MC-38 cells subcutaneously into the right flank. When tumors reached a volume of 50-100 mm³ (day 5) animals were divided in 2 cohorts and treatment was initiated:
Cohort 1 (7 animals): Isotype control (CTL IgG; BioxCell InVivo MAb Rat IgG2b, anti-KLH; Clone LTF-2, Cat #6E0090); 200 μg in 100 μL phosphate-buffered saline (PBS), intra-peritoneal (i.p.) on day 5, 7, 9, and 11.
Cohort 2 (6 animals): T22d35-Fc, 5 mg/kg in 100 μL PBS, i.p. on day 5, 9, 12, and 16.

Tumors were measured twice a week using digital calipers up to 15 days after commencing treatment. Tumor volumes were calculated from these measurements using a modified ellipsoidal formula (Tᵥₒₗ=π/6×(Length×Width×Width)) described previously (Tomayko et al., 1986).

### Results & Discussion

### Fusion Construct Design

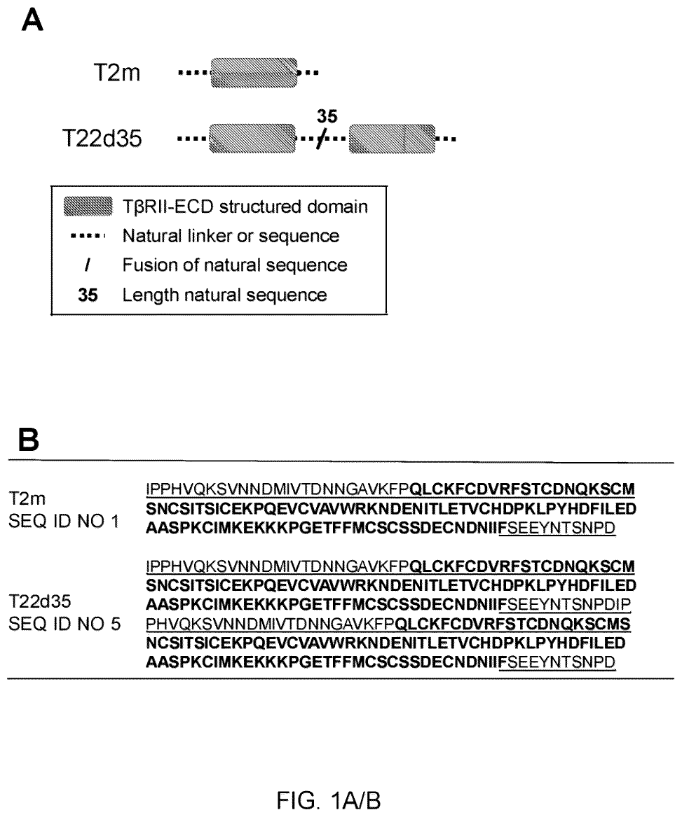

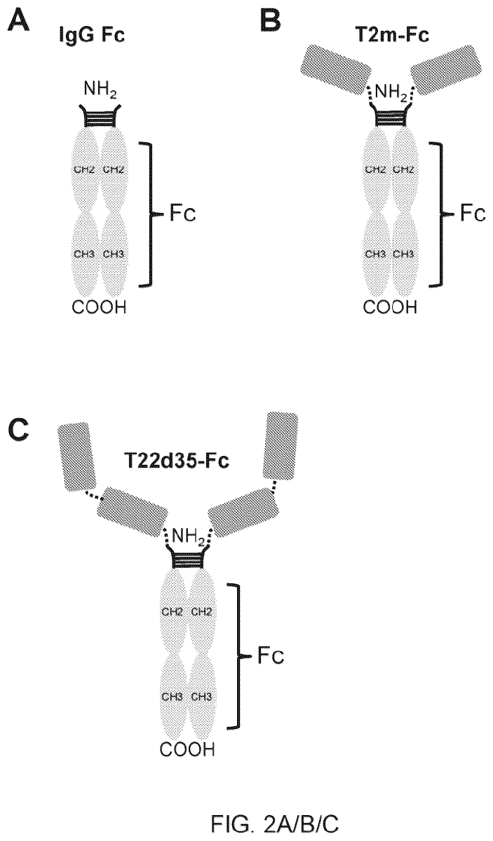

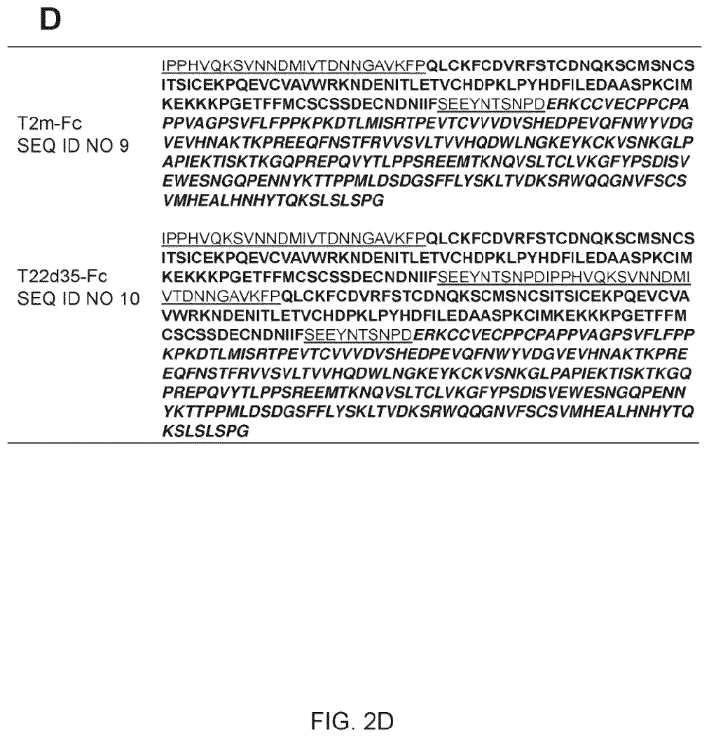

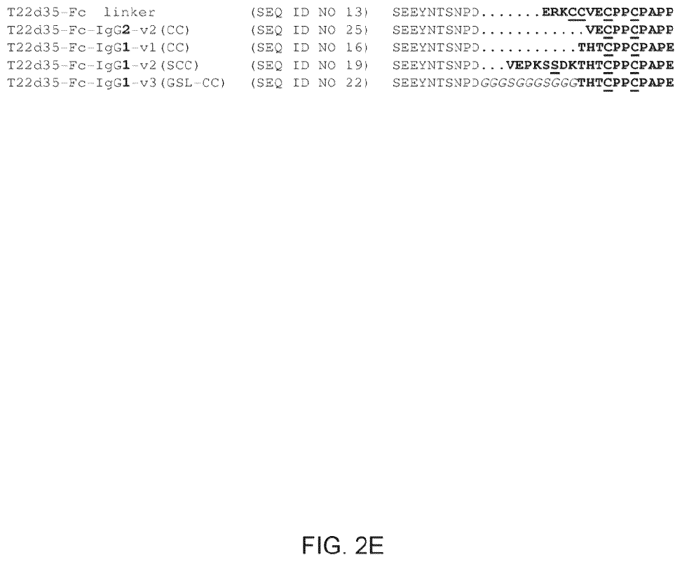

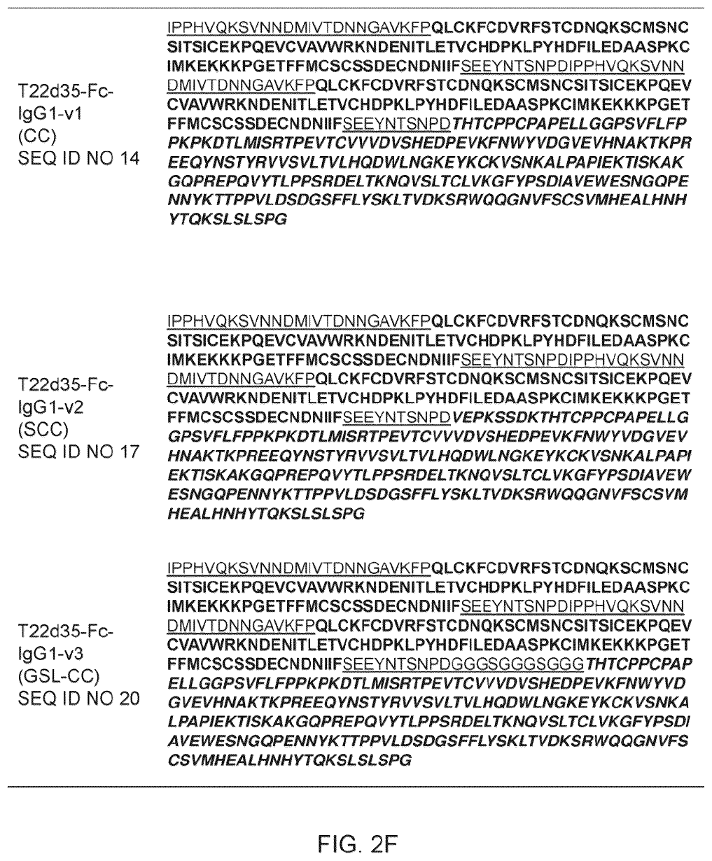

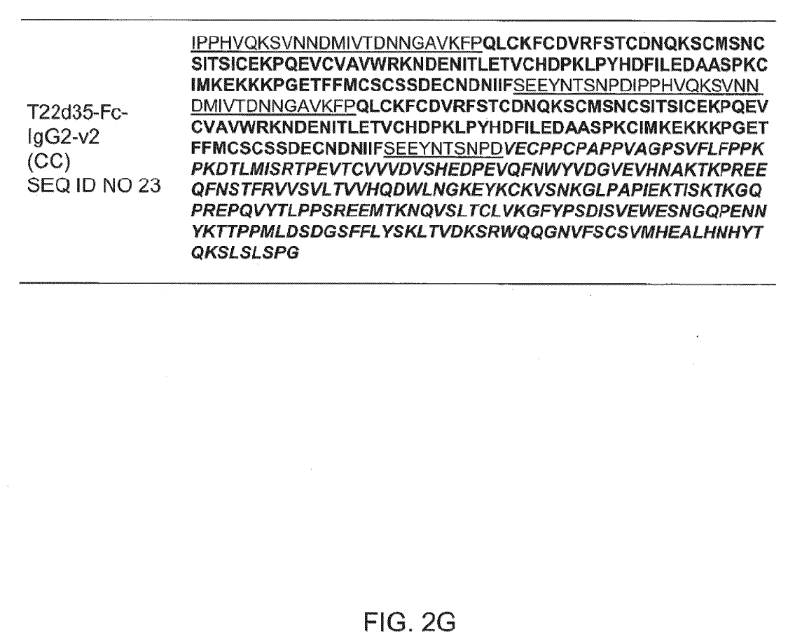

In order to generate TGF-β traps of interest, we fused the TβRII-ECD singlet (designated T2m) to another such singlet thereby forming the ectodomain doublet (designated T22d35) that was coupled to the N-termini of the heavy chains of a human (h)IgG2 Fc region and a human IgG1 Fc region. FIG. 1 shows schematics (FIG. 1A) and amino acid sequences (FIG. 1B) of the T2m and T22d35. These modules were fused to the N-termini of the heavy chains of an IgG Fc region (FIG. 2A) using several linker variations (FIG. 2E) in order to generate the T2m-Fc (FIG. 2B) and T22d35-Fc variants (FIG. 2C) fusions. The sequences of these fusions are shown in FIG. 2D. We also designed variants of T22d35-Fc that explore the number of cysteine residues in the hinge region of the Fc domain, different IgG isotypes (human IgG1 versus IgG2), and sequences of varying length and nature as linkers between T22d35 and the N-terminus of the Fc domain (FIGS. 2E, 2F & 2G). These variations aim at exploring and eventually optimizing the functional and manufacturability attributes of the T22d35-Fc design.

### Expression and Purification

### Purification of Transient CHO Material

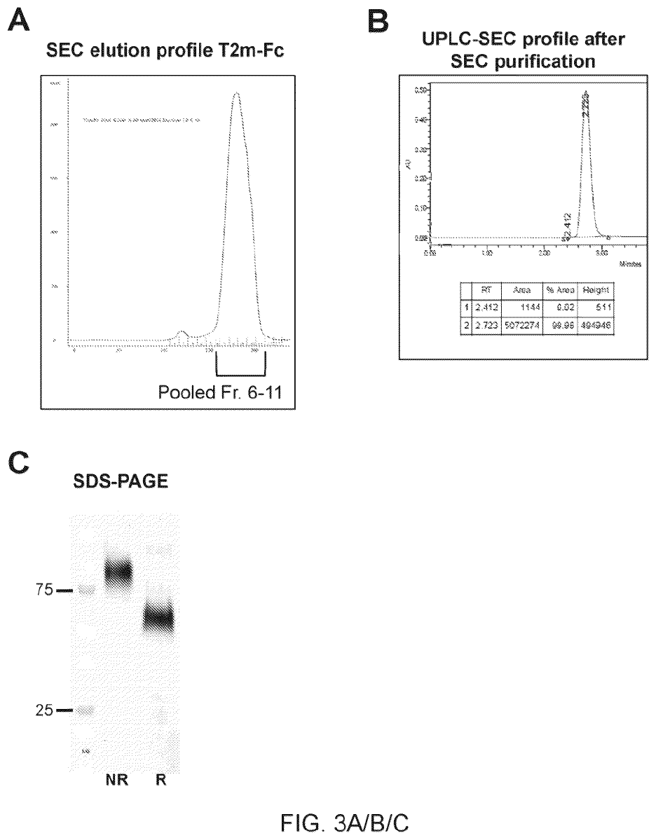

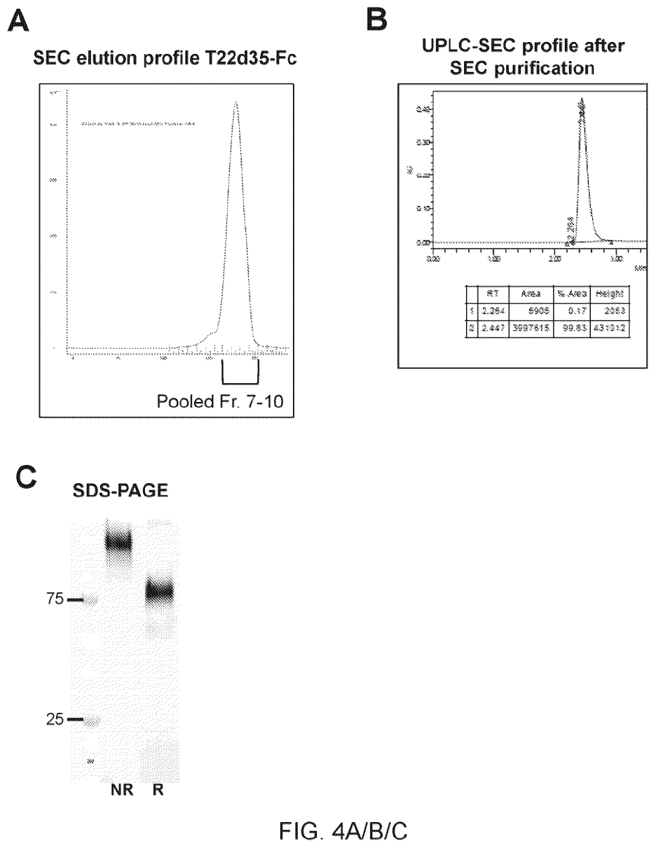

The respective fusion protein constructs were expressed transiently in CHO-3E7 cells (see Table 1) after which the conditioned medium was harvested and purified using a protein A affinity column, followed by preparative Size Exclusion Chromatography (SEC). SEC elution profiles of the T2m-Fc (FIG. 3A) and T22d35-Fc (FIG. 4A) showed that these fusion proteins are relatively pure and devoid of aggregates. Fractions 6-11 (T2m-Fc) and 7-10 (T22d35-Fc) were pooled and concentrated to 5.6 mg/mL (T2m-Fc) and 6.03 mg/mL (T22d35-Fc). The final yields were 267 mg and 168 mg for T2m-Fc and T22d35-Fc, respectively. The final products (indicated SEC pooled fractions) were shown to be >99% pure by UPLC-SEC (FIGS. 3B & 4B). SDS-PAGE assessment (FIGS. 3C & 4C, Sypro RUBY staining) shows the T2m-Fc and T22d35-Fc bands of ˜60 kDa and ˜90 kDa under reducing conditions, whereas bands of approximately 90 kDa and 150 kDa can be detected, representing the fully assembled and highly pure T2m-Fc and T22d35-Fc fusion proteins, respectively, under non-reducing conditions. An overview of the production and purification details can be found in Table 1. Together, these results demonstrate the good manufacturability of the T2m-Fc and T22d35-Fc fusion proteins.

TABLE 1
Production (transient pools) and purification
details of the T2m-Fc and T22d35-Fc fusion proteins.

| | T2m-Fc | T22d35-Fc |
|---|---|---|
| Cell line | CHO-3E7 | CHO-3E7 |
| production method | Transient | Transient |
| Production volume (L) | 2.5 | 4.6 |
| % GFP @ 24 hpt (Cellometer K2) | 38.5 | 41 |
| Production length | 15 | 15 |
| (days post transfection) | | |
| Average cell viability @ harvest (%) | 88.43 | 89.4 |
| Final volume (L; after 0.2 μm filtration) | 2.458 | 4.368 |
| Titre (mg/mL: pA-HPLC) | 139 | 54 |
| Maximum expected yield (mg) | 341 | 235 |
| Final yield (mg) | 267 | 168 |
| Recovery (%) | 78.30 | 71.49 |

### Purification of Stable CHO Pool Material

N- and C-terminally Fc-fused T22d35 variants were stably expressed in CHO^{BRI/rcTA} cells in order to compare their level of expression and some of their biophysical properties. The coding region of each variant was ligated into mammalian cell expression plasmids and, after transfection, an enriched pool of cells was selected that stably expressed each of the variants. The main difference between the variants can be found in the amino acid sequence composing the linker region that separates the T22d35 doublet from the Fc domain (in the case of the N-terminal fusions), while for the C-terminal Fc-fusions, the difference between each of the variants is at the extreme amino-terminus of the protein (Table 2).

TABLE 2
Description of amino acid variations in the linker region of the N- and C-terrninal Fc-
fused T22d35 fusions (Bold: natural linker sequence; italics: artificial linker sequence). The
paired cysteine residues in each of the variants are underlined.

| | | Relevant sequence differences |
|---|---|---|
| Variant ID | Fc Orientation/Isotype | (SEQ ID NOs: 35-42) |
| T22d35-Fc | N-terminal/IgG2 | T22d35........ERKCCVECPPCPAPP... |
| T22d35-Fc-IgG2-v2 | N-terminal/IgG2 | T22d35.............VECPPCPAPP... |
| T22d35-Fc-IgG1-v1 | N-terminal/IgG1 | T22d35............THTCPPCPAPE... |
| T22d35-Fc-IgG1-v2 | N-terminal/IgG1 | T22d35....VEPKSSDKTHTCPPCPAPE... |
| T22d35-Fc-IgG1-v3 | N-terminal/IgG1 | T22d35.GGGSGGGSGGGTHTCPPCPAPE... |
| Fc-IgG1-T22d35-v1 | C-terminal/IgG1 | PPCPAPE...T22d35 |
| Fc-IgG1-T22d35-v2 | C-terminal/IgG1 | DKTHTCPPCPAPE...T22d35 |
| Fc-IgG2-T22d35-v1 | C-terminal/IgG2 | VECPPCPAPP...T22d35 |

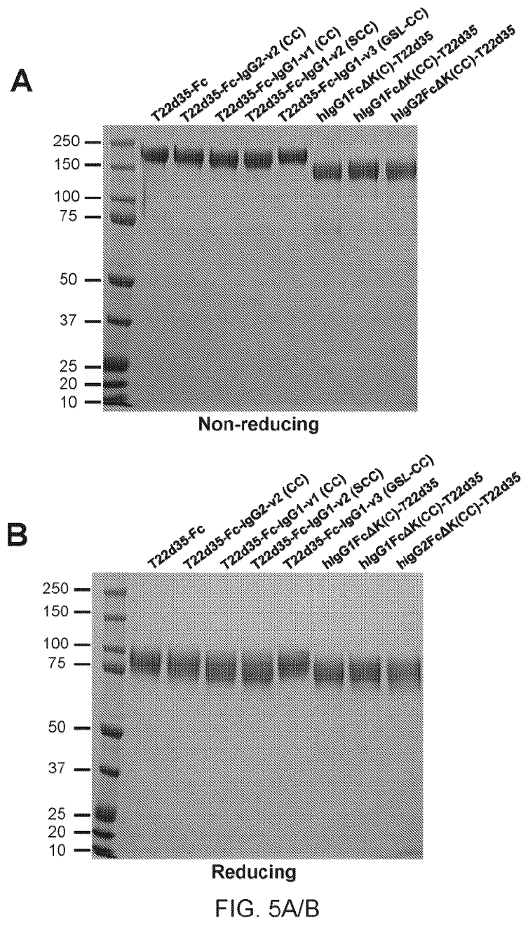

Fusion proteins were purified by protein A affinity and using 100 mM citrate (pH3.6) as the elution buffer. Eluted fusion protein samples were neutralized with 1 M HEPES then subjected to a buffer exchange to DPBS using Zeba spin columns (Table 3), while the integrity of several of the purified fusion proteins was assessed by SDS-PAGE (FIG. 5). Purification of each variant was similar. Although many of the properties were very similar between the variants, the potential for aggregation, which is indicative of improper folding, revealed some distinctions. Protein aggregation can be indicative of reduced conformational stability, and can result in decreased activity, efficacy or potency. Size-Exclusion Chromatography-High Performance Liquid Chromatography (SEC-HPLC) was used to determine the purity of each of the N- and C-terminal Fc-fused variants. This method allows for the accurate measurement of the percentage of intact monomeric species as well as the presence of impurities such as aggregates and/or degradation products. As shown in FIG. 6, a striking difference can be observed between T22d35 variants expressed as N-terminal Fc fusions and those expressed as C-terminal fusions. In particular, the percentage of intact monomer (FIG. 6A) was approximately 99% for all five N-terminal fusion variants (SEQ ID NO:10, SEQ ID NO:14, SEQ ID NO:17, SEQ ID NO:20, SEQ ID NO:23) whereas this was markedly lower for the three C-terminal fusions (SEQ ID NO:26, SEQ ID NO:27, SEQ ID NO:28). This significant decrease in the percentage of intact monomer results from the accumulation of increased higher molecular weight aggregates observed in all of the C-terminal Fc-fusions (FIG. 6B) as well as increased lower molecular weight fragments in two out of the three C-terminal Fc fusions (FIG. 6C). In addition, evaluation of the titers of the individual 500 mL productions and the average titers of the N-terminal Fc-fused and C-terminally Fc-fused T22d35 productions shows that the N-terminal Fc-fused T22d35 variants can be produced at higher yield compared to the C-terminal fusions (Table 4). Taken together, these results indicate that there are significant and unexpected advantages to expressing the T22d35 doublet at the N-terminus of moieties, such as the Fc portion of an immunoglobulin. Taken together these data demonstrate the enhanced manufacturability of the N-terminal Fc-fused T22d35 proteins.

TABLE 3
Overview of protein yields of the T22d35 variants after purification of 500 mL of stable
pool material.

| | T22d35- | T22d35- | T22d35- | T22d35- | T22d35- | hIgG1Fc | hIgG1Fc | hIgG2Fc |
|---|---|---|---|---|---|---|---|---|
| | Fc-IgG2 | Fc-IgG2- | Fc-IgG1- | Fc-IgG1- | Fc-IgG1- | ΔK(C)- | ΔK(CC)- | ΔK(CC)- |
| | (CCCC) | v2 (CC) | v1 (CC) | v2 (SCC) | v3 (GSL-CC) | T22d35 | T22d35 | T22d35 |
| Cell line | CHO- | CHO- | CHO- | CHO- | CHO- | CHO- | CHO- | CHO- |
| | 55E1 | 55E1 | 55E1 | 55E1 | 55E1 | 55E1 | 55E1 | 55E1 |
| Production method | Stable | Stable | Stable | Stable | Stable | Stable | Stable | Stable |
| | pool | pool | pool | pool | pool | pool | pool | pool |
| Production volume at start (L) | 0.5 | 0.5 | 0.5 | 0.5 | 0.5 | 0.5 | 0.5 | 0.5 |
| Production length (days post | 10 | 10 | 10 | 10 | 10 | 10 | 10 | 10 |
| induction) | | | | | | | | |
| Average cell viability @ harvest | 69.4 | 97 | 97.6 | 95.1 | 98 | 94.3 | 95.7 | 90.3 |
| (%) | | | | | | | | |
| Final volume (L; after 0.2 μm | 0.65 | 0.65 | 0.65 | 0.65 | 0.65 | 0.65 | 0.65 | 0.65 |
| filtration | | | | | | | | |
| Titer (mg/L: pA-HPLC) | 212 | 214 | 556 | 353 | 526 | 260 | 339 | 119 |
| Maximum expected yield (mg) | 150 | 150 | 150 | 150 | 150 | 150 | 150 | 150 |
| Final yield (mg) | 67.34 | 62.17 | 81.64 | 81.74 | 74.84 | 70.95 | 81.49 | 48.73 |
| Recovery (%) | 44.89 | 41.45 | 54.43 | 54.49 | 49.89 | 47.30 | 54.33 | 32.49 |

TABLE 4
Comparison of the titers of the individual N- and C-terminal Fc-fused T22d35 fusions

| | N-terminal Fc Fusions | | | C-terminal Fc Fusions | | | Improvement |
|---|---|---|---|---|---|---|---|
| | | | Average | | | Average | Titer (N/C- |
| | | Titer | Titer | | Titer | Titer | terminal Fc- |
| | Variant | (mg/mL) | (mg/mL) | Variant | (mg/mL) | (mg/mL) | fusion) |
| hIgG1 | T22d35-Fc-IgG1-v1 | 556 | 478 | hIgG1FcΔK(C)- | 260 | 300 | 63% |
| | (CC) | | | T22d35 | | | |
| | T22d35-Fc-IgG1-v2 | 353 | | hIgG1FcΔK(CC)- | 339 | | |
| | (SCC) | | | T22d35 | | | |
| | T22d35-Fc-IgG1-v3 | 526 | | | | | |
| | (GSL-CC) | | | | | | |
| hIgG2 | T22d35-Fc (CCCC) | 212 | 213 | hIgG2FcΔK(CC)- | 119 | 119 | 56% |
| | T22d35-Fc-IgG2-v2 | 214 | | T22d35 | | | |
| | (CC) | | | | | | |

### Functional In Vitro Assessment

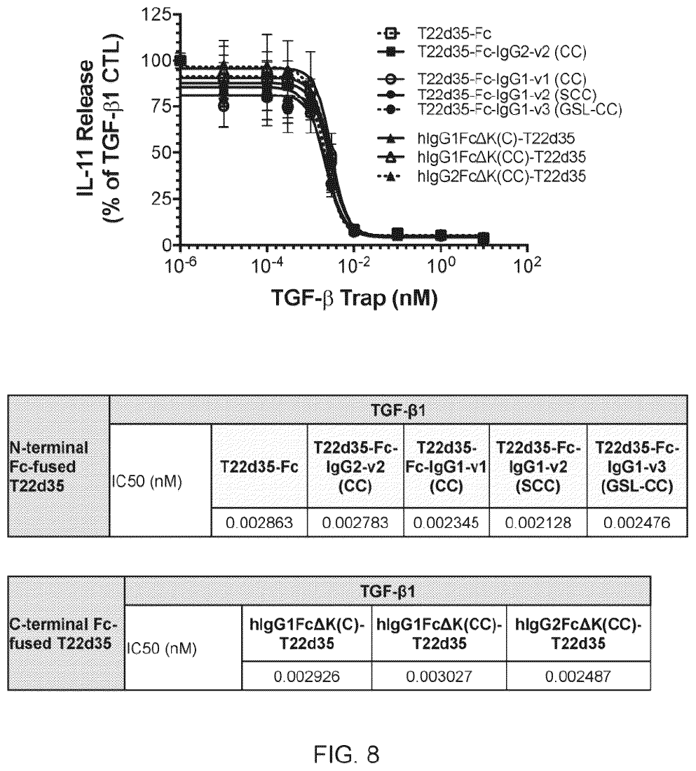

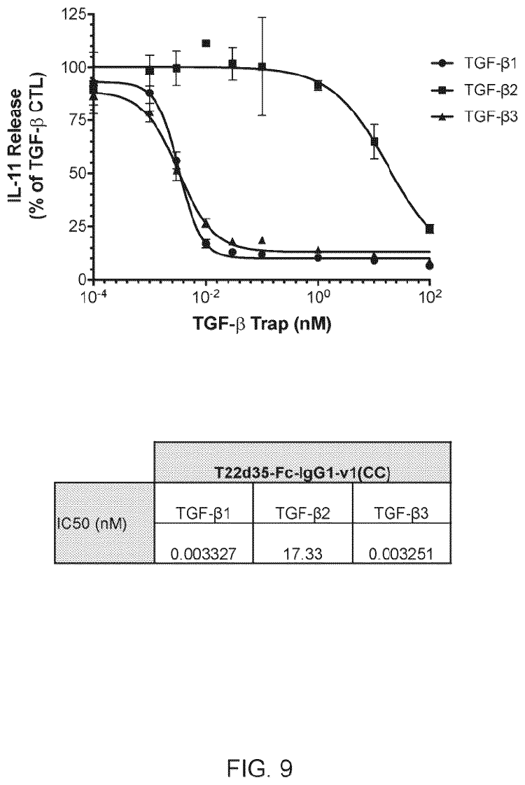

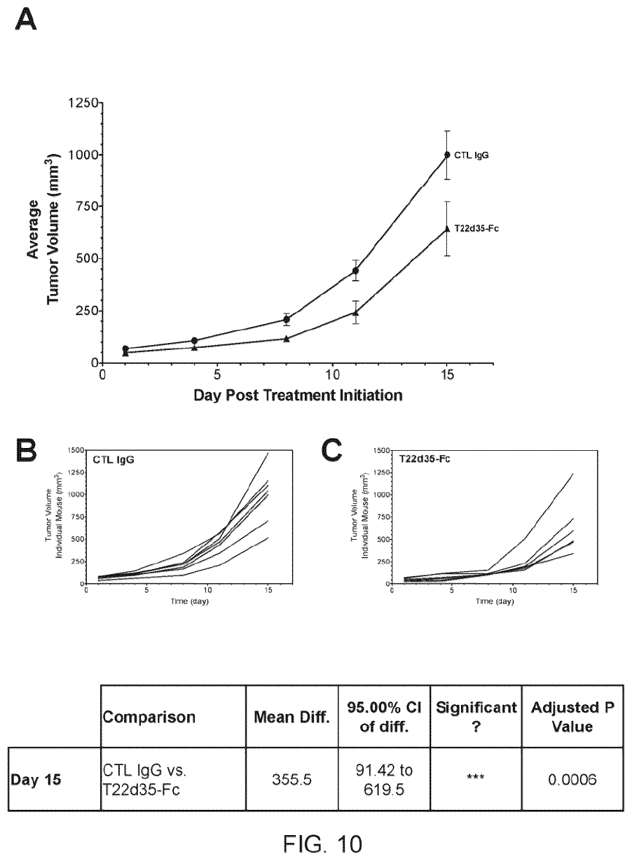

The A549 cell IL-11 release assay was used to compare TGF-β neutralization potencies of the T2m-Fc and T22d35-Fc fusion proteins to the non-Fc-fused T22d35 single chain doublet trap, as shown in FIG. 7A/B/C. This data shows that for all TGF-β isotypes the potency of T22d35-Fc is superior to that of T2m-Fc and the non-Fc-fused T22d35 single chain trap, with a calculated IC₅₀ (Table 5) of 0.003348 and 0.003908 nM for TGF-β1 and TGF-β3, respectively. These values demonstrate potencies that are at least 970-fold and at least 240-fold better than for T22d35 (IC₅₀=3.253 and 0.9491 nM, for TGF-β1 and TGF-β3, respectively), and 615-fold and 24-fold better than for T2m-Fc (IC₅₀=2.059 and 0.0943 nM, for TGF-β1 and TGF-β3, respectively). In addition, T22d35-Fc neutralizes TGF-β2, albeit to a much lesser extend than TGF-β1 and -β3. In contrast, TGF-β2 neutralization is not observed for either the T2m-Fc or the T22d35 single chain trap. It should be noted that, although the neutralization potency of the T22d35-Fc trap is similar for TGF-β1 and -β3, the T2m-Fc variant displayed a ˜22-fold higher neutralization potency for TGF-β3 compared to TGF-β1 (2.059 nM and 0.0943 nM, respectively). Evaluation of the additional N-terminal Fc-fused T22d35 fusions [T22d35-Fc-IgG2-v2 (CC), T22d35-Fc-IgG1-v1 (CC), T22d35-Fc-IgG1-v2 (SCC), and T22d35-Fc-IgG1-v3 (GSL-CC)] (FIG. 8, Table 6) showed that all of these fusions display comparable TGF-β1 neutralization potencies, which were very similar to the potency of T22d35-Fc. Additional evaluation of the T22d35-Fc-IgG1-v1 (CC) variant (FIG. 9) confirms that, in line with the T22d35-Fc variant, its neutralization potency for TGF-β1 and -β3 is very similar (IC₅₀=0.003327 nM and 0.003251 nM, respectively) whereas this potency is much lower for TGF-β2 (IC₅₀=17.33 nM).

TABLE 5
Overview of the statistical evaluation of the curves shown in Figure 5 using the 4-PL
algorithm ((log (inhibitor) vs. response − variable slope (four parameters)) available in Graphpad
Prism.

| | TGF-β1 | | | TGF-β2 | | | TGF-β3 | | |
|---|---|---|---|---|---|---|---|---|---|
| | T22d35 | T2m-Fc | T22d35-Fc | T22d35 | T2m-Fc | T22d35-Fc | T22d35 | T2m-Fc | T22d35-Fc |
| HillSlope | −1.236 | −1.088 | −1.991 | −0.08682 | ~ −16.05 | ~ −4.763 | −1.022 | −0.965 | −1.318 |
| IC50 (nM) | 3.253 | 2.059 | 0.003348 | None | None | ~10.58 | 0.9491 | 0.0943 | 0.003908 |
| R square | 0.9364 | 0.8918 | 0.9364 | — | — | 0.676 | 0.9258 | 0.8634 | 0.9624 |
| Outliers | 0 | 2 | 1 | — | — | — | — | — | — |
| (excluded, | | | | | | | | | |
| Q = 1%) | | | | | | | | | |

TABLE 6
Overview of the statistical evaluation of the curves shown in Figure 7 using the 4-PL
algorithm ((log (inhibitor) vs. response −variable slope (four parameters)) available in Graphpad
Prism.

| | TGF-β1 | | | | |
|---|---|---|---|---|---|
| | | T22d35-Fc-IgG2- | T22d35-Fc-IgG1- | T22d35-Fc-IgG1- | T22d35-Fc-IgG1- |
| | T22d35-Fc | v2 (CC) | v1 (CC) | v2 (SCC) | v3 (GSL-CC) |
| HillSlope | −2.25 | −2.13 | −2.056 | −2.063 | −2.655 |
| IC50 (nM) | 0.002863 | 0.002783 | 0.002345 | 0.002128 | 0.002476 |
| R square | 0.9805 | 0.9844 | 0.9422 | 0.9729 | 0.9464 |

### Functional In Vivo Assessment

The T22d35-Fc fusion protein (SEQ ID NO:10) was evaluated in vivo using a syngeneic MC-38 mouse colon carcinoma model (FIG. 9). The tumor growth in animals treated with the T22d35-Fc fusion was compared to the tumor growth in animals treated with a control IgG (CTL IgG). As shown in FIG. 9, no significant differences in tumor growth were observed up to day 11 post-treatment, however on day 15 a significant reduction in tumor growth can be observed in the tumor volume of animals treated with T22d35-Fc, when compared to the CTL IgG (Two-Way ANOVA). This data shows that administration of T22d35-Fc caused a significant inhibition in the growth of the MC-38 tumors compared to the group treated with the CTL IgG suggesting that blockage of TGF-β in vivo can abrogate the growth of tumors in this syngeneic model of colorectal cancer.

LISTING OF SEQUENCES

| SEQ | | |
|---|---|---|
| ID NO: | Sequence | Description |
| 1 | IPPHVQKSVNNDMIVTDNNGAVKFPQLCKFCDVRFSTCDNQKSC | TβRII-ECD |
| | MSNCSITSICEKPQEVCVAVWRKNDENITLETVCHDPKLPYHDFI | including the |
| | LEDAASPKCIMKEKKKPGETFFMCSCSSDECNDNIIFSEEYNTSN | structure |
| | PD | domain and its |
| | | natural linkers |
| | | (also termed |
| | | T2m) |
| 2 | IPPHVQKSVNNDMIVTDNNGAVKFPQLCKFCDVRFSTCDNQKSC | TβRII-ECD |
| | MSNCSITSICEKPQEVCVAVWRKNDENITLETVCHDPKLPYHDFI | structured |
| | LEDAASPKCIMKEKKKPGETFFMCSCSSDECNDNIIF | domain with its |
| | | natural N- |
| | | terminal linker |
| 3 | QLCKFCDVRFSTCDNQKSCMSNCSITSICEKPQEVCVAVWRKN | TβRII-ECD |
| | DENITLETVCHDPKLPYHDFILEDAASPKCIMKEKKKPGETFFMC | structured |
| | SCSSDECNDNIIFSEEYNTSNPD | domain with its |
| | | with natural C- |
| | | terminal linker |
| 4 | QLCKFCDVRFSTCDNQKSCMSNCSITSICEKPQEVCVAVWRKN | TβRII-ECD |
| | DENITLETVCHDPKLPYHDFILEDAASPKCIMKEKKKPGETFFMC | structured |
| | SCSSDECNDNIIF | domain |
| 5 | IPPHVQKSVNNDMIVTDNNGAVKFPQLCKFCDVRFSTCDNQKSC | TβRII-ECD- |
| | MSNCSITSICEKPQEVCVAVWRKNDENITLETVCHDPKLPYHDFI | TβRII-ECD fused |
| | LEDAASPKCIMKEKKKPGETFFMCSCSSDECNDNIIFSEEYNTSN | dimer including |
| | PDIPPHVQKSVNNDMIVTDNNGAVKFPQLCKFCDVRFSTCDNQK | structured |
| | SCMSNCSITSICEKPQEVCVAVWRKNDENITLETVCHDPKLPYH | domains and |
| | DFILEDAASPKCIMKEKKKPGETFFMCSCSSDECNDNIIFSEEYN | natural linkers |
| | TSNPD | (also termed |
| | | T22d35) |
| 6 | SEEYNTSNPDIPPHVQKSVNNDMIVTDNNGAVKF | TβRII-ECD |
| | | natural linker |
| 7 | IPPHVQKSVNNDMIVTDNNGAVKF | TβRII-ECD N- |
| | | terminal natural |
| | | linker |
| 8 | SEEYNTSNPD | TβRII-ECD C- |
| | | terminal natural |
| | | linker |
| 9 | IPPHVQKSVNNDMIVTDNNGAVKFPQLCKFCDVRFSTCDNQKSC | T2m-Fc fusion of |
| | MSNCSITSICEKPQEVCVAVWRKNDENITLETVCHDPKLPYHDFI | T2m with the |
| | LEDAASPKCIMKEKKKPGETFFMCSCSSDECNDNIIFSEEYNTSN | hlgG2Fc(CCCC) Fc |
| | PDERKCCVECPPCPAPPVAGPSVFLFPPKPKDTLMISRTPEVTCV | Fc region |
| | VVDVSHEDPEVQFNWYVDGVEVHNAKTKPREEQFNSTFRVVSVLT | |
| | VVHQDWLNGKEYKCKVSNKGLPAPIEKTISKTKGQPREPQVYTLP | |
| | PSREEMTKNQVSLTCLVKGFYPSDISVEWESNGQPENNYKTTPPM | |
| | LDSDGSFFLYSKLTVDKSRWQQGNVFSCSVMHEALHNHYTQKSLS | |
| | LSPG | |
| 10 | IPPHVQKSVNNDMIVTDNNGAVKFPQLCKFCDVRFSTCDNQKSC | T22d35-Fc |
| | MSNCSITSICEKPQEVCVAVWRKNDENITLETVCHDPKLPYHDFI | fusion f T22d35 |
| | LEDAASPKCIMKEKKKPGETFFMCSCSSDECNDNIIFSEEYNTSN | hlgG2Fc(CCCC) Fc |
| | PDIPPHVQKSVNNDMIVTDNNGAVKFPQLCKFCDVRFSTCDNQK | Fc region |
| | SCMSNCSITSICEKPQEVCVAVWRKNDENITLETVCHDPKLPYH | |
| | DFILEDAASPKCIMKEKKKPGETFFMCSCSSDECNDNIIFSEEYN | |
| | TSNPDERKCCVECPPCPAPPVAGPSVFLFPPKPKDTLMISRTPEV | |
| | TCVVVDVSHEDPEVQFNQYVDGVEVHNAKTKPREEQFNSTFRVVS | |
| | VLTVVHQDWLNGKEYKCKVSNKGLPAPIEKTISKTKGQPREPQVY | |
| | TLPPSREEMTKNQVSLTCLVKGFYPSDISVEWESNGQPENNYKTT | |
| | PPMLDSDGSFFLYSKLTVDKSRWQQGNVFSCSVMHEALHNHYTQK | |
| | SLSLSPG | |
| 11 | MDWTWRILFLVAAATGTHA | signal peptide |
| 12 | ERKCCVECPPCPAPPVAGPSVFLFPPKPKDTLMISRTPEVTCVVV | hlgG2Fc(CCCC) Fc |
| | DVSHEDPEVQFNWYVDGVEVHNAKTKPREEQFNSTFRVVSLVLTV | region variant |
| | VHQDWLNGKEYKCKVSNKGLPAPIEKTISKTKGQPREPQVYTLPP | |
| | SREEMTKNQVSLTCLVKGFYPSDISVEWESNGQPENNYKTTPPML | |
| | DSDGSFFLYSKLTVDKSRWQQGNVFSCSVMHEALHNHYTQKSLSL | |
| | SPG | |
| 13 | SEEYNTSNPDERKCCVECPPCPAPP | T22d35-Fc |
| | | natural linker |
| | | with the |
| | | hlgG2Fc(CCCC) |
| | | hinge region |
| 14 | IPPHVQKSVNNDMIVTDNNGAVKFPQLCKFCDVRFSTCDNQKSC | T22d35-Fc-IgG1- |
| | MSNCSITSICEKPQEVCVAVWRKNDENITLETVCHDPKLPYHDFI | v1(CC) usion of |
| | LEDAASPKCIMKEKKKPGETFFMCSCSSDECNDNIIFSEEYNTSN | hlgG1Fc(CC) Fc |
| | PDIPPHVQKSVNNDMIVTDNNGAVKFPQLCKFCDVRFSTCDNQK | region |
| | SCMSNCSITSICEKPQEVCVAVWRKNDENITLETVCHDPKLPYH | |
| | DFILEDAASPKCIMKEKKKPGETFFMCSCSSDECNDNIIFSEEYN | |
| | TSNPDTHTCPPCPAPELLGGPSVFLFPPKPKDTLMISRTPEVTCV | |
| | VVDVSHEDPEVKFNWYVDGVEVHNAKTKPREEQYNSTYRVVSVLT | |
| | VLHQDWLNGKEYKCKVSNKALPAPIEKISKAKGQPREPQVYTLPP | |
| | SRDELTKNQVSLTCLVKGFYPSDIAVEWESNGQPENNYKTTPPVL | |
| | DSDGSFFLYSKLTVDKSRWQQGNVFSCSVMHEALHNHYTQKSLSL | |
| | SPG | |
| 15 | THTCPPCPAPELLGGPSVFLFPPKPKDTLMISRTPEVTCVVVDVS | hlgG1Fc(CC) Fc |
| | HEDPEVKFNWYVDGVEVHNAKTKPREEQYNSTYRVVSVLTVLHQD | region variant |
| | WLNGKEYKCKVSNKALPAPIEKTISKAKGQPREPQVYTLPPSRDE | |
| | LTKNQVSLTCLVKGFYPSDIAVEWESNGQPENNYKTTPPVLDSDG | |
| | SFFLYSKLTVDKSRWQQGNVFSCSVMHEALHNHYTQKSLSLSPG | |
| 16 | SEEYNTSNPDTHTCPPCPAPE | T22d35-Fc-IgG1- |
| | | v1(CC) natural |
| | | linker with the |
| | | hlgG1Fc(CC) |
| | | hinge region |
| 17 | IPPHVQKSVNNDMIVTDNNGAVKFPQLCKFCDVRFSTCDNQKSC | T22d35-Fc-IgG1- |
| | MSNCSITSICEKPQEVCVAVWRKNDENITLETVCHDPKLPYHDFI | v2(SCC) fusion |
| | LEDAASPKCIMKEKKKPGETFFMCSCSSDECNDNIIFSEEYNTSN | of T22d35 with |
| | PDIPPHVQKSVNNDMIVTDNNGAVKFPQLCKFCDVRFSTCDNQK | the |
| | SCMSNCSITSICEKPQEVCVAVWRKNDENITLETVCHDPKLPYH | hlgG1Fc(SCC) |
| | DFILEDAASPKCIMKEKKKPGETFFMCSCSSDECNDNIIFSEEYN | Fc region |
| | TSNPDVEPKSSDKTHTCPPCPAPELLGGPSVFLFPPKPKDTLMIS | |
| | RTPEVTCVVVDVSHEDPEVKFNWYVDGVEVHNAKTKPREEQYNST | |
| | YRVVSVLTVLHQDWLNGKEYKCKVSNKALPAPIEKTISKAKGQPR | |
| | EPQVYTLPPSRDELTKNQVSLTCLVKGFYPSDIAVEWESNGQPEN | |
| | NYKTTPPVLDSDGSFFLYSKLTVDKSRWQWQGNVFSCSVMHEALH | |
| | NHYTQKSLSLSPG | |
| 18 | VEPKSSDKTHTCPPCPAPELLGGPSVFLFPPKPKDTLMISRTPEV | hlgG1Fc(SCC) Fc |
| | TCVVVDVSHEDPEVKFNWYVDGVEVHNAKTKPREEQYNSTYRVVS | Fc region variant |
| | VLTVLHQDWLNGKEYKCKVSNKALPAPIEKTISKAKGQPREPQVY | |
| | TLPPSRDELTKNQVSLTCLVKGFYPSDIAVEWESNGQPENNYKTT | |
| | PPVLDSDGSFFLYSKLTVDKSRWQQGNVFSCSVMHEALHNHYTQK | |
| | SLSLSPB | |
| 19 | SEEYNTSNPDVEPKSSDKTHTCPPCPAPE | T22d35-Fc-IgG1- |
| | | v2(SCC) natural |
| | | linker with the |
| | | hlgG1Fc(SCC) |
| | | hinge region |
| 20 | IPPHVQKSVNNDMIVTDNNGAVKFPQLCKFCDVRFSTCDNQKSC | T22d35-Fc-IgG1- |
| | MSNCSITSICEKPQEVCVAVWRKNDENITLETVCHDPKLPYHDFI | v3(GSL-CC) |
| | LEDAASPKCIMKEKKKPGETFFMCSCSSDECNDNIIFSEEYNTSN | with the |
| | PDIPPHVQKSVNNDMIVTDNNGAVKFPQLCKFCDVRFSTCDNQK | fusion of T22d35 |
| | SCMSNCSITSICEKPQEVCVAVWRKNDENITLETVCHDPKLPYH | with the |
| | DFILEDAASPKCIMKEKKKPGETFFMCSCSSDECNDNIIFSEEYN | hlgG1Fc(CC) Fc |
| | TSNPDGGGSGGGSGGGTHTCPPCPAPELLGGPSVFLFPPKPKDTL | region and |
| | MISRTPEVTCVVVDVSHEDPEVKFNWYVDGVEVHNAKTKPREEQY | including an |
| | NSTYRVVSVLTVLHQDWLNGKEYKCKVSNKALPAPIEKTISKAKG | artificial GS |
| | QPREPQVYTLPPSRDELTKNQVSLTCLVKGFYPSDIAVEWESNGQ | linker |
| | PENNYKTTPPVLDSDGSFFLYSKLTVDKSRWQQGNVFSCSVMHEA | |
| | LHNHYTQKSLSLSPG | |
| 21 | GGGSGGGSGGG | Artificial GS |
| | | linker of the |
| | | T22d35-Fc-IgG1- |
| | | v3(GSL-CC) |
| | | fusion |
| 22 | SEEYNTSNPDGGGSGGGSGGGTHTCPPCPAPE | T22d35-Fc-IgG1- |
| | | v3(GSL-CC) |
| | | linker including |
| | | natural and |
| | | artificial |
| | | sequences and |
| | | HLGg1Fc(CC) |
| | | hinge region |
| 23 | IPPHVQKSVNNDMIVTDNNGAVKFPQLCKFCDVRFSTCDNQKSC | T22d35-Fc-IgG2- |
| | MSNCSITSICEKPQEVCVAVWRKNDENITLETVCHDPKLPYHDFI | v2(CC) fusion of |
| | LEDAASPKCIMKEKKKPGETFFMCSCSSDECNDNIIFSEEYNTSN | T22d35 with the |
| | PDIPPHVQKSVNNDMIVTDNNGAVKFPQLCKFCDVRFSTCDNQK | hlgG2Fc(cc) Fc |
| | SCMSNCSITSICEKPQEVCVAVWRKNDENITLETVCHDPKLPYH | region |
| | DFILEDAASPKCIMKEKKKPGETFFMCSCSSDECNDNIIFSEEYN | |
| | TSNPD | |
| 24 | VECPPCPAPPVAGPSVFLFPPKPKDTLMISRTPEVTCVVVDVSHE | hlgF2Fc(CC) Fc |
| | DPEVQFNWYVDGVEVHNAKTKPREEQFNSTFRVVSVLTVVHQDWL | region variant |
| | NGKEYKCKVSNKGLPAPIEKTISKTKGQPREPQVYTLPPSREEMT | |
| | KNQVSLTCLVKGFYPSDISVEWESNGQPENNYKTTPPMLDSDGSF | |
| | FLYSKLTVDKSRWQQGNVFSCSVMHEALHNHYTQKSLSLSPG | |
| 25 | SEEYNTSNPDVECPPCPAPP | T22d35-Fc-IgG2- |
| | | v2(CC) natural |
| | | linker with the |
| | | hlgG2Fc(CC) |
| | | hinge region |
| 26 | PPCPAPELLGGPSVFLFPPKPKDTLMISRTPEVTCVVVDVSHEDP | hlgG1FcΔK(C)- |
| | EVKFNWYYDGVEVHNAKTKPREEQYNSTYRVVSVLTVLHQDWLNG | T22d35 |
| | KEYKCKVSNKALPAPIEKTISKAKGQPREPQVYTLPPSRDELTKN | |
| | QVSLTCLVKGFYPSDIAVEWESNGQPENNYKTTPPVLDSDGSFFL | |
| | YSKLTVDKSRWQQGNVFSCSVMHEALHNHYTQKSLSLSPG | |
| | IPPHVQKSVNNDMIVTDNNGAVKFPQLCKFCDVRFSTCDNQK | |
| | SCMSNCSITSICEKPQEVCVAVWRKNDENITLETVCHDPKLPYH | |
| | DFILEDAASPKCIMKEKKKPGETFFMCSCSSDECNDNIIFSEEYN | |
| | TSNPDIPPHVQKSVNNDMIVTDNNGAVKFPQLCKFCDVRFSTCD | |
| | NQKSCMSNCSITSICEKPQEVCVAVWRKNDENITLETYCHDPKL | |
| | PYHDFILEDAASPKCIMKEKKKPGETFFMCSCSSDECNDNIIFSE | |
| | EYNTSNPD | |
| 27 | DKTHTCPPCPAPELLGGPSVFLFPPKPKDTLMISRTPEVTCVVVD | hlgG1FcΔK(CC)- |
| | VSHEDPEVKFNWYVDGVEVHNAKTKPREEQYNSTYRVVSVLTVLH | T22d35 |
| | QDWLNGKEYKCKVSNKALPAPIEKTISKAKGQPREPQVYTLPPSR | |
| | DELTKNQVSLTCLVKGFYPSDIAVEWESNGQPENNYKTTPPVLDS | |
| | DGSFFLYSKLTVDKSRWQQGNVFSCSVMHEALHNHYTQKSLSLSPG | |
| | IPPHVQKSVNNDMIVTDNNGAVKFPQLCKFCDVRFS | |
| | TCDNQKSCMSNCSITSICEKPQEVCVAVWRKNDENITLETYCHD | |
| | PKLPYHDFILEDAASPKCIMKEKKKPGETFFMCSCSSDECNDNII | |
| | FSEEYNTSNPDIPPHVQKSVNNDMIVTDNNGAVKFPQLCKFCDV | |
| | RFSTCDNQKSCMSNCSITSICEKPQEVCVAVWRKNDENITLETV | |
| | CHDPKLPYHDFILEDAASPKCIMKEKKKPGETFFMCSCSSDECN | |
| | DNIIFSEEYNTSNPD | |
| 28 | VECPPCPAPPVAGPSVFLFPPKPKDTLMISRTPEVTCVVVDVSH | hlgG2FcΔK(CC)- |
| | EDPEVQFNWYVDGVEVHNAKTKPREEQFNSTFRVVSVLTVVHQD | T22d35 |
| | WLNGKEYKCKVSNKGLPAPIEKTISKTKGQPREPQVYTLPPSRE | |
| | EMTKNQVSLTCLVKGFYPSDISVEWESNGQPENNYKTTPPMLDS | |
| | DGSFFLYSKLTVDKSRWQQGNVFSCSVMHEALHNHYTQKSLSLSPG | |
| | IPPHVQKSVNNDMIVIDNNGAVKFPQLCKFCDVRFSTCD | |
| | NQKSCMSNCSITSICEKPQEVCVAVWRKNDENITLETVCHDPKL | |
| | PYHDFILEDAASPKCIMKEKKKPGETFFMCSCSSDECNDNIIFSE | |
| | EYNTSNPDIPPHVQKSVNNDMIVTDNNGAVKFPQLCKFCDVRFS | |
| | TCDNQKSCMSNCSITSICEKPQEVCVAVWRKNDENITLETYCHD | |
| | PKLPYHDFILEDAASPKCIMKEKKKPGETFFMCSCSSDECNDNII | |
| | FSEEYNTSNPD | |
| 29 | ATGGATTGGACCTGGAGAATCCTCTTCCTTGTAGCAGCAGCAA | Nucleic acid |
| | CAGGTACACATGCTATCCCTCCTCATGTTCAAAAGTCCGTTAA | sequence |
| | CAACGACATGATCGTCACCGATAACAACGGTGCTGTCAAGTTC | encoding |
| | CCACAACTCTGTAAGTTCTGCGATGTGCGTTTCTCCACATGTG | T22d35-Fc in |
| | ATAACCAGAAGTCCTGTATGAGCAACTGCTCAATCACCTCCAT | secretable form |
| | CTGCGAAAAGCCACAAGAGGTATGCGTAGCTGTATGGCGAAA | |
| | GAACGATGAAAACATCACCCTGGAAACCGTCTGTCACGATCCA | |
| | AAGCTCCCATACCATGATTTCATCCTGGAAGACGCAGCTTCTC | |
| | CAAAGTGTATCATGAAGGAGAAGAAGAAGCCCGGTGAAACCTT | |
| | CTTCATGTGCTCCTGTTCCTCAGATGAATGCAACGATAACATC | |
| | ATCTTCTCCGAGGAGTACAACACCTCCAACCCAGATATCCCTC | |
| | CACACGTTCAGAAGTCCGTAAACAATGACATGATTGTGACCGA | |
| | CAACAACGGGGCTGTTAAGTTCCCACAGCTCTGTAAGTTTTGC | |
| | GACGTTAGGITCAGCACCIGTGATAATCAGAAGAGCTGCATGT | |
| | CCAACTGCAGCATCACCAGTATTTGCGAGAAGCCTCAAGAAGT | |
| | GTGTGTCGCTGTTTGGAGAAAGAACGACGAAAACATAACCCTG | |
| | GAGACCGTTTGCCACGATCCAAAACTCCCATATCACGATTTCA | |
| | TTCTGGAGGACGCCGCCAGTCCTAAATGTATAATGAAAGAGAA | |
| | GAAGAAACCAGGGGAGACCTTCTTTATGTGCAGCTGCAGCAG | |
| | CGACGAGTGTAACGATAATATAATTTTTAGCGAGGAGTATAATA | |
| | CAAGCAATCCCGACGAGCGCAAGTGCTGCGTCGAGTGCCCTC | |
| | CATGCCCTGCCCCTCCTGTTGCCGGACCTAGTGTGTTTTTGTT | |
| | TCCTCCTAAACCTAAAGATACACTCATGATTAGCAGGACACCT | |
| | GAGGTGACATGTGTCGTCGTGGACGTGAGTCATGAAGACCCC | |
| | GAAGTGCAGTTTAATTGGTATGTCGACGGAGTCGAAGTCCATA | |
| | ATGCCAAAACTAAACCAAGGGAAGAACAGTTTAATTCAACTTTT | |
| | CGCGTGGTCTCTGTGCTGACTGTGGTGCACCAGGACTGGCTT | |
| | AATGGAAAGGAATACAAGTGTAAGGTGAGTAATAAGGGCCTGC | |
| | CCGCCCCCATTGAAAAAACTATTAGTAAGACTAAAGGGCAGCC | |
| | CCGAGAGCCCCAGGTGTATACTTTGCCCCCCTCTCGGGAGGA | |
| | GATGACTAAAAATCAGGTGAGTCTTACATGTCTTGTGAAAGGA | |
| | TTTTACCCCTCTGACATTTCAGTGGAGTGGGAGTCTAATGGCC | |
| | AGCCCGAGAATAATTACAAAACTACTCCCCCCATGTTGGACTC | |
| | TGACGGCTCATTTTTCTTGTACTCTAAACTGACAGTGGACAAAA | |
| | GTCGGTGGCAGCAGGGCAATGTGTTTTCTTGTTCAGTGATGCA | |
| | CGAGGCCCTGCATAATCACTATACACAGAAATCTCTGTCTCTG | |
| | TCACCCGGCTGATGA | |
| 30 | ATGGACTGGACCTGGAGAATCCTGTTCCTGGTGGCTGCTGCT | Nucleic acid |
| | ACCGGAACACACGCTATCCCCCCTCATGTGCAGAAGTCCGTG | sequence |
| | AACAATGACATGATCGTGACAGATAACAATGGCGCCGTGAAGT | encoding |
| | TTCCTCAGCTGTGCAAGTTCTGTGACGTGAGGTTTAGCACCTG | T22d35-Fc-IgG1- |
| | CGATAACCAGAAGTCCTGCATGAGCAATTGTTCTATCACATCC | v1(CC) in |
| | ATCTGCGAGAAGCCACAGGAGGTGTGCGTGGCCGTGTGGCG | secretable form |
| | GAAGAACGACGAGAATATCACCCTGGAGACAGTGTGCCACGA | |
| | TCCTAAGCTGCCATACCATGACTTCATCCTGGAGGATGCTGCC | |
| | TCTCCCAAGTGTATCATGAAGGAGAAGAAGAAGCCTGGCGAG | |
| | ACATTCTTCATGTGCTCCTGTTCCAGCGACGAGTGCAACGATA | |
| | ATATCATCTTCAGCGAGGAGTATAACACCTCTAATCCAGATATC | |
| | CCACCCCACGTGCAGAAGTCTGTCAATAACGATATGATTGTCA | |
| | CAGATAACAATGGCGCTGTGAAGTTTCCCCAGCTGTGCAAATT | |
| | TTGTGACGTGAGATTTTCCACCTGTGATAACCAGAAGAGCTGC | |
| | ATGTCTAATTGTTCCATCACATCTATTTGTGAAAAACCTCAGGA | |
| | AGTGTGCGTGGCCGTGTGGAGAAAAAATGATGAAAACATCAC | |
| | CCTGGAGACAGTGTGCCATGATCCCAAGCTGCCTTATCACGA | |
| | CTTCATCCTGGAAGACGCTGCCAGCCCAAAATGCATTATGAAA | |
| | GAGAAGAAGAAGCCCGGTGAGACATTCTTCATGTGCAGCTGTT | |
| | CTTCTGATGAATGTAACGATAATATCATCTTTTCCGAGGAGTAT | |
| | AACACAAGCAATCCCGACACCCACACATGCCCTCCATGTCCAG | |
| | CTCCTGAGCTGCTGGGAGGACCTAGCGTGTTCCTGTTTCCCC | |
| | CTAAGCCAAAGGATACCCTGATGATCAGCAGGACCCCCGAGG | |
| | TGACATGCGTGGTGGTGGACGTGTCTCACGAGGACCCCGAGG | |
| | TGAAGTTTAACTGGTACGTGGACGGCGTGGAGGTGCATAATG | |
| | CCAAGACCAAGCCTAGGGAGGAGCAGTACAACTCTACCTATC | |
| | GGGTGGTGTCCGTGCTGACAGTGCTGCATCAGGATTGGCTGA | |
| | ACGGCAAGGAGTATAAGTGCAAGGTGTCCAATAAGGCTCTGC | |
| | CAGCCCCCATTGAGAAGACCATCAGCAAGGCTAAGGGCCAGC | |
| | CAAGAGAGCCCCAGGTGTACACACTGCCACCCTCTCGCGACG | |
| | AGCTGACCAAGAACCAGGTGTCCCTGACATGTCTGGTGAAGG | |
| | GCTTCTATCCTTCCGATATCGCTGTGGAGTGGGAGAGCAACG | |
| | GACAGCCAGAGAACAATTACAAGACCACACCTCCAGTGCTGG | |
| | ACTCTGATGGCTCCTTCTTTCTGTATAGCAAGCTGACCGTGGA | |
| | CAAGTCTAGGTGGCAGCAGGGCAACGTGTTTAGCTGTTCTGT | |
| | GATGCATGAGGCCCTGCACAATCATTACACACAGAAGTCCCTG | |
| | AGCCTGTCTCCTGGC | |
| 31 | ATGGACTGGACCTGGAGAATCCTGTTCCTGGTGGCTGCTGCT | Nucleic acid |
| | ACCGGAACACACGCTATCCCCCCTCATGTGCAGAAGTCTGTG | sequence |
| | AACAATGACATGATCGTGACAGATAACAATGGCGCCGTGAAGT | encoding |
| | TTCCCCAGCTGTGCAAGTTCTGTGACGTGAGGTTTTCCACCTG | T22d35-Fc-IgG1- |
| | CGATAACCAGAAGTCTTGCATGTCCAATTGTAGCATCACATCT | v2(SCC) in |
| | ATCTGCGAGAAGCCTCAGGAGGTGTGCGTGGCCGTGTGGCG | secretable form |
| | GAAGAACGACGAGAATATCACCCTGGAGACAGTGTGCCACGA | |
| | TCCTAAGCTGCCATACCATGACTTCATCCTGGAGGATGCTGCC | |
| | AGCCCAAAGTGTATCATGAAGGAGAAGAAGAAGCCCGGCGAG | |
| | ACATTCTTCATGTGCTCTTGTTCCAGCGACGAGTGCAACGATA | |
| | ATATCATCTTCTCCGAGGAGTATAACACCAGCAATCCTGACAT | |
| | CCCACCCCACGTGCAGAAGAGCGTCAATAACGATATGATTGTC | |
| | ACAGATAACAATGGCGCTGTGAAGTTTCCACAGCTGTGCAAAT | |
| | TTTGTGACGTGAGATTTTCTACCTGTGATAACCAGAAGTCCTG | |
| | CATGAGCAATTGTTCTATCACATCCATCTGCGAGAAGCCACAG | |
| | GAAGTGTGCGTGGCCGTGTGGAGAAAAAATGATGAAAACATC | |
| | ACCCTGGAGACAGTGTGCCATGATCCCAAGCTGCCTTATCAC | |
| | GACTTCATCCTGGAAGACGCTGCCTCCCCTAAATGCATTATGA | |
| | AAGAGAAGAAGAAGCCAGGTGAGACATTCTTCATGTGCAGCT | |
| | GTTCTTCTGATGAGTGCAACGATAACATCATCTTTTCTGAGGA | |
| | GTACAACACATCCAATCCTGACGTGGAGCCAAAGAGCTCTGAT | |
| | AAGACCCACACATGCCCTCCATGTCCAGCTCCTGAGCTGCTG | |
| | GGAGGACCATCCGTGTTCCTGTTTCCACCTAAGCCTAAGGACA | |
| | CCCTGATGATCTCCAGGACCCCAGAGGTGACATGCGTGGTGG | |
| | TGGACGTGAGCCACGAGGACCCCGAGGTGAAGTTTAACTGGT | |
| | ACGTGGATGGCGTGGAGGTGCATAATGCCAAGACCAAGCCAA | |
| | GGGAGGAGCAGTACAACAGCACCTATCGGGTGGTGTCTGTGC | |
| | TGACAGTGCTGCATCAGGACTGGCTGAACGGCAAGGAGTATA | |
| | AGTGCAAGGTGTCTAATAAGGCTCTGCCAGCCCCCATCGAGA | |
| | AGACCATCTCCAAGGCTAAGGGCCAGCCAAGAGAGCCCCAGG | |
| | TGTACACACTGCCACCCAGCCGCGACGAGCTGACCAAGAACC | |
| | AGGTGTCTCTGACATGTCTGGTGAAGGGCTTCTATCCCTCTGA | |
| | TATCGCTGTGGAGTGGGAGTCCAACGGACAGCCTGAGAACAA | |
| | TTACAAGACCACACCTCCAGTGCTGGACAGCGATGGCTCTTTC | |
| | TTTCTGTATTCCAAGCTGACCGTGGATAAGAGCAGGTGGCAGC | |
| | AGGGCAACGTGTTTTCCTGTAGCGTGATGCATGAGGCCCTGC | |
| | ACAATCATTACACACAGAAGTCTCTGTCCCTGAGCCCTGGC | |
| 32 | ATGGATTGGACCTGGAGAATCCTGTTCCTGGTGGCTGCTGCTA | Nucleic acid |
| | CCGGAACACACGCTATCCCCCCTCATGTGCAGAAGTCTGTGA | sequence |
| | ACAATGACATGATCGTGACAGATAACAATGGCGCCGTGAAGTT | encoding |
| | TCCTCAGCTGTGCAAGTTCTGTGACGTGAGGTTTTCCACCTGC | T22d35-Fc-IgG10 |
| | GATAACCAGAAGTCCTGCATGAGCAATTGTTCTATCACATCCA | v3(GSL-CC) in |
| | TCTGCGAGAAGCCACAGGAGGTGTGCGTGGCCGTGTGGCGG | secretable form |
| | AAGAACGACGAGAATATCACCCTGGAGACAGTGTGCCACGAT | |
| | CCTAAGCTGCCATACCATGACTTCATCCTGGAGGATGCTGCCA | |
| | GCCCCAAGTGTATCATGAAGGAGAAGAAGAAGCCTGGCGAGA | |
| | CATTCTTCATGTGCTCTTGTTCCAGCGACGAGTGCAACGATAA | |
| | TATCATCTTCTCCGAGGAGTATAACACCAGCAATCCAGACATC | |
| | CCACCCCACGTGCAGAAGAGCGTCAATAACGATATGATTGTCA | |
| | CAGATAACAATGGCGCTGTGAAGITTCCOCAGCTGTGCAAATT | |
| | TTGTGACGTGAGATTTTCTACCTGTGATAACCAGAAGAGCTGC | |
| | ATGTCTAATTGTTCCATCACATCTATTTGTGAAAAACCTCAGGA | |
| | AGTGTGCGTGGCCGTGTGGAGAAAAAATGATGAAAACATCAC | |
| | CCTGGAGACAGTGTGCCATGATCCCAAGCTGCCTTATCACGA | |
| | CTTCATCCTGGAAGACGCTGCCTCCCCAAAATGCATTATGAAA | |
| | GAGAAGAAGAAGCCCGGTGAGACATTCTTCATGTGCAGCTGTT | |
| | CTTCTGATGAGTGCAACGATAACATCATCTTTTCTGAGGAGTA | |
| | CAACACATCCAATCCTGACGGAGGAGGCAGCGGAGGAGGCTC | |
| | TGGAGGCGGCACCCACACATGCCCTCCATGTCCAGCTCCTGA | |
| | GCTGCTGGGAGGACCTTCCGTGTTCCTGTTTCCCCCTAAGCC | |
| | AAAGGACACCCTGATGATCTCCAGGACCCCCGAGGTGACATG | |
| | CGTGGTGGTGGACGTGAGCCACGAGGACCCCGAGGTGAAGT | |
| | TTAACTGGTACGTGGATGGCGTGGAGGTGCATAATGCCAAGA | |
| | CCAAGCCAAGGGAGGAGCAGTACAACAGCACCTATCGGGTGG | |
| | TGTCTGTGCTGACAGTGCTGCATCAGGATTGGCTGAACGGCA | |
| | AGGAGTATAAGTGCAAGGTGTCTAATAAGGCTCTGCCAGCCC | |
| | CCATTGAGAAGACCATCTCCAAGGCTAAGGGCCAGCCAAGAG | |
| | AGCCCCAGGTGTACACACTGCCACCCAGCCGCGACGAGCTGA | |
| | CCAAGAACCAGGTGTCTCTGACATGTCTGGTGAAGGGCTTCTA | |
| | TCCTTCTGATATCGCTGTGGAGTGGGAGTCCAACGGACAGCC | |
| | AGAGAACAATTACAAGACCACACCTCCAGTGCTGGACTCTGAT | |
| | GGCTCCTTCTTTCTGTATTCCAAGCTGACCGTGGACAAGAGCA | |
| | GGTGGCAGCAGGGCAACGTGTTTAGCTGTTCTGTGATGCATG | |
| | AGGCCCTGCACAATCATTACACACAGAAGTCCCTGAGCCTGTC | |
| | TCCTGGC | |
| 33 | ATGGATTGGACCTGGAGAATCCTCTTCCTTGTAGCAGCAGCAA | Nucleic acid |
| | CAGGTACACATGCTATCCCTCCTCATGTTCAAAAGTCCGTTAA | sequence |
| | CAACGACATGATCGTCACCGATAACAACGGTGCTGTCAAGTTC | encoding |
| | CCACAACTCTGTAAGTTCTGCGATGTGCGTTTCTCCACATGTG | T22d357-Fc-IgG2- |
| | ATAACCAGAAGTCCTGTATGAGCAACTGCTCAATCACCTCCAT | v2(CC) in |
| | CTGCGAAAAGCCACAAGAGGTATGCGTAGCTGTATGGCGAAA | secretable form |
| | GAACGATGAAAACATCACCCTGGAAACCGTCTGTCACGATCCA | |
| | AAGCTCCCATACCATGATTTCATCCTGGAAGACGCAGCTTCTC | |
| | CAAAGTGTATCATGAAGGAGAAGAAGAAGCCCGGTGAAACCTT | |
| | CTTCATGTGCTCCTGTTCCTCAGATGAATGCAACGATAACATC | |
| | ATCTTCTCCGAGGAGTACAACACCTCCAACCCAGATATCCCTC | |
| | CACACGTTCAGAAGTCCGTAAACAATGACATGATTGTGACCGA | |
| | CAACAACGGGGCTGTTAAGTTCCCACAGCTCTGTAAGTTTTGC | |
| | GACGTTAGGTTCAGCACCTGTGATAATCAGAAGAGCTGCATGT | |
| | CCAACTGCAGCATCACCAGTATTTGCGAGAAGCCTCAAGAAGT | |
| | GTGTGTCGCTGTTTGGAGAAAGAACGACGAAAACATAACCCTG | |
| | GAGACCGTTTGCCACGATCCAAAACTCCCATATCACGATTTCA | |
| | TTCTGGAGGACGCCGCCAGTCCTAAATGTATAATGAAAGAGAA | |
| | GAAGAAACCAGGGGAGACCTTCTTTATGTGCAGCTGCAGCAG | |
| | CGACGAGTGTAACGATAATATAATTTTTAGCGAGGAGTATAATA | |
| | CAAGCAATCCCGACGTCGAGTGCCCTCCATGCCCTGCCCCTC | |
| | CTGTTGCCGGACCTAGTGTGTTTTTGTTTCCTCCTAAACCTAAA | |
| | GATACACTCATGATTAGCAGGACACCTGAGGTGACATGTGTCG | |
| | TCGTGGACGTGAGTCATGAAGACCCCGAAGTGCAGTTTAATTG | |
| | GTATGTCGACGGAGTCGAAGTCCATAATGCCAAAACTAAACCA | |
| | AGGGAAGAACAGTTTAATTCAACTTTTCGCGTGGTCTCTGTGC | |
| | TGACTGTGGTGCACCAGGACTGGCTTAATGGAAAGGAATACAA | |
| | GTGTAAGGTGAGTAATAAGGGCCTGCCCGCCCCCATTGAAAA | |
| | AACTATTAGTAAGACTAAAGGGCAGCCCCGAGAGCCCCAGGT | |
| | GTATACTTTGCCCCCCTCTCGGGAGGAGATGACTAAAAATCAG | |
| | GTGAGTCTTACATGTCTTGTGAAAGGATTTTACCCCTCTGACAT | |
| | TTCAGTGGAGTGGGAGTCTAATGGCCAGCCCGAGAATAATTAC | |
| | AAAACTACTCCCCCCATGTTGGACTCTGACGGCTCATTTTTCTT | |
| | GTACTCTAAACTGACAGTGGACAAAAGTCGGTGGCAGCAGGG | |
| | CAATGTGTTTTCTTGTTCAGTGATGCACGAGGCCCTGCATAAT | |
| | CACTATACACAGAAATCTCTGTCTCTGTCACCCGGCTGATGA | |
| 34 | (GGGGS)n | glycine-serine |
| | | linker (GSL) - |
| | | may be repeated |
| | | between 1 and |
| | | 50 times |
| 35 | ERKCCVECPPCPAPP | amino acid |
| | | variations in |
| | | the linker |
| | | region of the |
| | | N- and C- |
| | | terminal FC- |
| | | fused T22d35 |
| | | fusions |
| 36 | VECPPCPAPP | amino acid |
| | | variations in |
| | | the linker |
| | | region of the |
| | | N- and C- |
| | | terminal FC- |
| | | fused T22d35 |
| | | fusions |
| 37 | THTCPPCPAPE | amino acid |
| | | variations in |
| | | the linker |
| | | region of the |
| | | N- and C- |
| | | terminal FC- |
| | | fused T22d35 |
| | | fusions |
| 38 | VEPKSSDKTHTCPPCPAPE | amino acid |
| | | variations in |
| | | the linker |
| | | region of the |
| | | N- and C- |
| | | terminal FC- |
| | | fused T22d35 |
| | | fusions |
| 39 | GGGSGGGSGGGTHTCPPCPAPE | amino acid |
| | | variations in |
| | | the linker |
| | | region of the |
| | | N- and C- |
| | | terminal FC- |
| | | fused T22d35 |
| | | fusions |
| 40 | PPCPAPE | amino acid |
| | | variations in |
| | | the linker |
| | | region of the |
| | | N- and C- |
| | | terminal FC- |
| | | fused T22d35 |
| | | fusions |
| 41 | DKTHTCPPCPAPE | amino acid |
| | | variations in |
| | | the linker |
| | | region of the |
| | | N- and C- |
| | | terminal FC- |
| | | fused T22d35 |
| | | fusions |
| 42 | VECPPCPAPP | amino acid |
| | | variations in |
| | | the linker |
| | | region of the |
| | | N- and C- |
| | | terminal FC- |
| | | fused T22d35 |
| | | fusions |

### REFERENCES

All patents, patent applications and publications referred to throughout the application are listed below.
Arteaga C L (2006) Inhibition of TGFβeta signaling in cancer therapy. Curr Opin Genet Dev 16: 30-37
De Crescenzo G, Grothe S, Zwaagstra J, Tsang M, O'Connor-McCourt M D (2001) Real-time monitoring of the interactions of transforming growth factor-beta (TGF-beta) isoforms with latency-associated protein and the ectodomains of the TGF-beta type II and III receptors reveals different kinetic models and stoichiometries of binding. J Biol Chem 276: 29632-29643
Durocher Y, Perret S, Kamen A (2002) High-level and high-throughput recombinant protein production by transient transfection of suspension-growing human 293-EBNA1 cells. Nucleic Acids Res 30: E9
Economides A N, Carpenter L R, Rudge J S, Wong V, Koehler-Stec E M, Hartnett C, Pyles E A, Xu X, Daly T J, Young M R, Fandl J P, Lee F, Carver S, McNay J, Bailey K, Ramakanth S, Hutabarat R, Huang T T, Radziejewski C, Yancopoulos G D, Stahl N (2003) Cytokine traps: multi-component, high-affinity blockers of cytokine action. Nat Med 9: 47-52
Eisenberg D, Schwarz E, Komaromy M, Wall R (1984) Analysis of membrane and surface protein sequences with the hydrophobic moment plot. J Mol Biol 179: 125-142
Gajewski T F (2015) The Next Hurdle in Cancer Immunotherapy: Overcoming the Non-T-Cell-Inflamed Tumor Microenvironment. Semin Oncol 42: 663-671
Garberg P, Ball M, Borg N, Cecchelli R, Fenart L, Hurst R D, Lindmark T, Mabondzo A, Nilsson J E, Raub T J, Stanimirovic D, Terasaki T, Oberg J O, Osterberg T (2005) In vitro models for the blood-brain barrier. Toxicol In Vitro 19: 299-334
Hahn T, Akporiaye E T (2006) Targeting transforming growth factor beta to enhance cancer immunotherapy. Curr Oncol 13: 141-143
Haqqani A S, Caram-Salas N, Ding W, Brunette E, Delaney C E, Baumann E, Boileau E, Stanimirovic D (2013) Multiplexed evaluation of serum and CSF pharmacokinetics of brain-targeting single-domain antibodies using a NanoLC-SRM-ILIS method. Mol Pharm 10: 1542-1556
Hawinkels L J, Ten Dijke P (2011) Exploring anti-TGF-beta therapies in cancer and fibrosis. Growth Factors 29: 140-152
Holash J, Davis S, Papadopoulos N, Croll S D, Ho L, Russell M, Boland P, Leidich R, Hylton D, Burova E, loffe E, Huang T, Radziejewski C, Bailey K, Fandl J P, Daly T, Wiegand S J, Yancopoulos G D, Rudge J S (2002) VEGF-Trap: a VEGF blocker with potent antitumor effects. Proc Natl Acad Sci U S A 99: 11393-11398
Jin P, Zhang J, Beryt M, Turin L, Brdlik C, Feng Y, Bai X, Liu J, Jorgensen B, Shepard H M (2009) Rational optimization of a bispecific ligand trap targeting EGF receptor family ligands. Mol Med 15: 11-20
Li M O, Wan Y Y, Sanjabi S, Robertson A K, Flavell R A (2006) Transforming growth factor-beta regulation of immune responses. Annu Rev Immuno124: 99-146
Massague J, Blain S W, Lo R S (2000) TGFβeta signaling in growth control, cancer, and heritable disorders. Cell 103: 295-309
Mourskaia A A, Northey J J, Siegel P M (2007) Targeting aberrant TGF-beta signaling in pre-clinical models of cancer. Anticancer Agents Med Chem 7: 504-514
Rodgarkia-Dara C, Vejda S, Erlach N, Losert A, Bursch W, Berger W, Schulte-Hermann R, Grusch M (2006) The activin axis in liver biology and disease. Mutat Res 613: 123-137
Santarpia M, Gonzalez-Cao M, Viteri S, Karachaliou N, Altavilla G, Rosell R (2015) Programmed cell death protein-1/programmed cell death ligand-1 pathway inhibition and predictive biomarkers: understanding transforming growth factor-beta role. Transl Lung Cancer Res 4: 728-742
Thiery J P, Acloque H, Huang R Y, Nieto M A (2009) Epithelial-mesenchymal transitions in development and disease. Cell 139: 871-890
Wojtowicz-Praga S (2003) Reversal of tumor-induced immunosuppression by TGF-beta inhibitors. Invest New Drugs 21: 21-32
Yang L, Pang Y, Moses H L (2010) TGF-beta and immune cells: an important regulatory axis in the tumor microenvironment and progression. Trends Immunol 31: 220-227
Yang X, Ambrogelly A (2014) Enlarging the repertoire of therapeutic monoclonal antibodies platforms: domesticating half molecule exchange to produce stable IgG4 and IgG1 bispecific antibodies. Curr Opin Biotechnol 30: 225-229
Zheng X, Koropatnick J, Chen D, Velenosi T, Ling H, Zhang X, Jiang N, Navarro B, Ichim TE, Urquhart B, Min W (2013) Silencing IDO in dendritic cells: a novel approach to enhance cancer immunotherapy in a murine breast cancer model. Int J Cancer 132: 967-977
Zwaagstra J C, Sulea T, Baardsnes J, Lenferink A E, Collins C, Cantin C, Paul-Roc B, Grothe S, Hossain S, Richer L P, L'Abbe D, Tom R, Cass B, Durocher Y, O'Connor-McCourt M D (2012) Engineering and therapeutic application of single-chain bivalent TGF-beta family traps. Mol Cancer Thar 11: 1477-1487
WO/1995/04069
WO/2004/076670
WO 2008/113185
WO 2010/031168
U.S. Pat. No. 8,815,247
U.S. Pat. No. 6,277,7375
US2015/0225483
WO01/83525;
WO2005/028517;
WO2008/113185;
WO2008/157367;
WO2010/003118;
WO2010/099219;
WO2012/071649;
WO2012/142515;
WO2013/000234;
U.S. Pat. No. 5,693,607;
US2005/0203022;
US2007/0244042;
U.S. Pat. No. 8,318,135;
U.S. Pat. No. 8,658,135;
U.S. Pat. No. 8,815,247;
US2015/0225483; and
US2015/0056199

1-31. (canceled)

32. A polypeptide comprising from N-terminus to C-terminus: (i) a first TGF-β receptor ectodomain (TβR-ECD); (ii) a second TβR-ECD; and (iii) a second constant domain (CH2) and/or third constant domain (CH3) of an antibody heavy chain;
wherein the polypeptide inhibits TGF-β1 and TGF-β3 activity with at least 100-fold greater potency than a non-Fc fused TβR-ECD doublet.
